(19) United States
(12) Patent Application Publication
(10) Pub. No.: US 2004/0132715 A1
(43) Pub. Date: Jul. 8, 2004

(54) Method to treat allergic rhinitis

(76) Inventors: Paul J. Dunford, San Diego, CA (US); James P. Edwards, San Diego, CA (US); Lars Karlsson, La Jolla, CA (US); Wai-Ping Leung, San Diego, CA (US); Robin L. Thurmond, San Diego, CA (US); Jianmei Wei, San Diego, CA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/656,498

(22) Filed: Sep. 5, 2003

Related U.S. Application Data
(60) Provisional application No. 60/408,579, filed on Sep. 6, 2002.

(51) Int. Cl. A61K 31/553; A61K 31/54; A61K 31/554; A61K 31/5377; A61K 31/496

(52) U.S. Cl. 514/211.15; 514/228.2; 514/234.2; 514/254.02; 514/254.06

(57) ABSTRACT

A method to treat allergic rhinitis is disclosed in which patients are administered certain indolyl compounds.

### FIELD OF THE INVENTION

The invention relates to novel, pharmaceutically active, fused heterocyclic compounds and methods of using them to treat or prevent disorders and conditions mediated by the histamine H₄ receptor.

### BACKGROUND OF THE INVENTION

Histamine was first identified as a hormone (G. Barger and H. H. Dale, J. Physiol. (London) 1910, 41:19-59) and has since been demonstrated to play a major role in a variety of physiological processes, including the inflammatory “triple response” via H₁ receptors (A. S. F. Ash and H. O. Schild, Br. J. Pharmac. Chemother. 1966, 27:427-439), gastric acid secretion via H₂ receptors (J. W. Black et al., Nature 1972, 236:385-390), and neurotransmitter release in the central nervous system via H₃ receptors (J.-M. Arrang et al., Nature 1983, 302:832-837) (for review see S. J. Hill et al., Pharmacol Rev. 1997, 49(3):253-278). All three histamine receptor subtypes have been demonstrated to be members of the superfamily of G protein-coupled receptors (I. Gantz et al., Proc. Natl. Acad. Sci. U.S.A. 1991, 88:429-433; T. W. Lovenberg et al., Mol. Pharmacol. 1999, 55(6):1101-1107; M. Yamashita et al., Proc. Natl. Acad. Sci. U.S.A. 1991, 88:11515-11519). There are, however, additional functions of histamine that have been reported, for which no receptor has been identified. For example, in 1994, Raible et al. demonstrated that histamine and R-α-methylhistamine could activate calcium mobilization in human eosinophils (D. G. Raible et al., Am. J. Respir. Crit. Care Med. 1994, 149:1506-1511). These responses were blocked by the H₃-receptor antagonist thioperamide. However, R-α-methylhistamine was significantly less potent than histamine, which was not consistent with the involvement of known H₃ receptor subtypes. Therefore, Raible et al. hypothesized the existence of a novel histamine receptor on eosinophils that was non-H₁, non-H₂, and non-H₃. Most recently several groups (T. Oda et al., J. Biol. Chem. 2000, 275(47):36781-36786; C. Liu et al., Mol. Pharmacol. 2001, 59(3):420-426; T. Nguyen et al., Mol. Pharmacol. 2001, 59(3):427-433; Y. Zhu et al., Mol. Pharmacol. 2001, 59(3):434-441; K. L. Morse et al., J. Pharmacol. Exp. Ther. 2001, 296(3):1058-1066) have identified and characterized a fourth histamine receptor subtype, the H₄ receptor. This receptor is a 390 amino acid, seven-transmembrane, G protein-coupled receptor with approximately 40% homology to the histamine H₃ receptor. In contrast to the H₃ receptor, which is primarily located in the brain, the H₄ receptor is expressed at greater levels in neutrophils and mast cells, among other cells, as reported by Morse et al. (see above).

Events that elicit the inflammatory response include physical stimulation (including trauma), chemical stimulation, infection, and invasion by a foreign body. The inflammatory response is characterized by pain, increased temperature, redness, swelling, reduced function, or a combination of these. Many conditions, such as allergies, asthma, chronic obstructed pulmonary disease (COPD), atherosclerosis, and autoimmune diseases, including rheumatoid arthritis and lupus, are characterized by excessive or prolonged inflammation. Inhibition of leukocyte recruitment can provide significant therapeutic value. Inflammatory diseases or inflammation-mediated diseases or conditions include, but are not limited to, acute inflammation, allergic inflammation, and chronic inflammation.

Mast cell de-granulation (exocytosis) leads to an inflammatory response that may be initially characterized by a histamine-modulated wheal and flare reaction. A wide variety of immunological (e.g., allergens or antibodies) and non-immunological (e.g., chemical) stimuli may cause the activation, recruitment, and de-granulation of mast cells. Mast cell activation initiates allergic (H₁) inflammatory responses, which in turn cause the recruitment of other effector cells that further contribute to the inflammatory response. The histamine H2 receptors modulate gastric acid secretion, and the histamine H3 receptors affect neurotransmitter release in the central nervous system.

Examples of textbooks on the subject of inflammation include J. I. Gallin and R. Snyderman, Inflammation: Basic Principles and Clinical Correlates, 3^{rd} Edition, (Lippincott Williams & Wilkins, Philadelphia, 1999); V. Stvrtinova, J. Jakubovsky and I. Hulin, “Inflammation and Fever”, Pathophysiology Principles of Diseases (Textbook for Medical Students, Academic Press, 1995); Cecil et al., Textbook Of Medicine, 18^{th} Edition (W. B. Saunders Company, 1988); and Steadmans Medical Dictionary.

A summary of the present invention follows.

### SUMMARY OF THE INVENTION

The invention features a compound of formula (I) wherein:

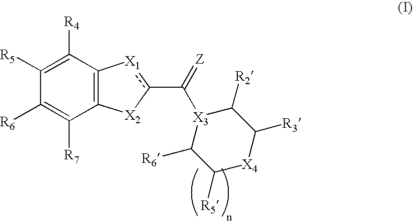

Wherein R₁ is Rₐ, RₐR_{b}—, Rₐ—O—R_{b}—, or (R_{c})(R_{d})N—R_{b}—, where Rₐ is H, cyano, —(C═O)N(R_{c})(R_{d}), —C(═NH)(NH₂), C₁₋₁₀ alkyl, C₃₋₈ alkenyl, C₃₋₈ cycloalkyl, C₂₋₅ heterocyclic radical, or phenyl; where R_{b} is C₁₋₈ alkylene, C₂₋₈ alkenylene, C₃₋₈ cycloalkylene, bivalent C₃₋₈ heterocyclic radical, or phenylene; and R_{c} and R_{d} are each independently H, C₁₋₈ alkyl, C₂₋₈ alkenyl, C₃₋₈ cycloalkyl, or phenyl;

R_{2′} is H, methyl, ethyl, NRₚR_{q}, —(CO)NRₚR_{q}, —(CO)ORᵣ, —CH₂NRₚR_{q}, or CH₂ORᵣ; where Rₚ, R_{q}, and Rᵣ are independently selected from C₁₋₆ alkyl, C₃₋₆ cycloalkyl, phenyl; (C₃₋₆ cycloalkyl)(C₁₋₂ alkylene), benzyl or phenethyl; or Rₚ and R_{q} taken together with the nitrogen to which they are attached, form a 4-7 membered heterocyclic ring with 0 or 1 additional heteroatoms selected from O, S, and N;

R_{3′} is H, methyl, ethyl, NRₛRₜ, —(CO)NRₛRₜ, —(CO)ORᵤ, —CH₂NRₛRₜ, or CH₂ORᵤ; where Rₛ, Rₜ, and Rᵤ are independently selected from C₁₋₆ alkyl, C₃₋₆ cycloalkyl, phenyl; (C₃₋₆ cycloalkyl)(C₁₋₂ alkylene), benzyl or phenethyl; or Rₛ and Rₜ taken together with the nitrogen to which they are attached, form a 4-7 membered heterocyclic ring with 0 or 1 additional heteroatoms selected from O, S, and N;

R_{5′} is methyl, ethyl, or H;

R_{6′} is methyl, ethyl, or H;

R_{7′} is methyl, ethyl, or H;

X₄ is NR₁ or S;

X₁ is CR₃;

R₃ is F, Cl, Br, CHO, R_{f}, R_{f}R_{g}—, R_{f}—O—R_{g}—, or (Rₕ)(Rᵢ)N—R_{g}—, where R_{f} is H, C₁₋₆ alkyl, C₂₋₆ alkenyl, C₃₋₆ cycloalkyl, C₂₋₅ heterocyclic radical, or phenyl; where R_{g} is C₁₋₆ alkylene, C₂₋₆ alkenylene, C₃₋₆ cycloalkylene, bivalent C₃₋₆ heterocyclic radical, or phenylene; and Rₕ and Rᵢ are each independently H, C₁₋₆ alkyl, C₂₋₆ alkenyl, C₃₋₆ cycloalkyl, or phenyl;

X₂ is NRₑ or O; Rₑ is H or C₁₋₆ alkyl;

X₃ is N;

Z is ═O or ═S;

each of R₄ and R₆ is independently H, F, Cl, Br, I, COOH, OH, nitro, amino, cyano, C₁₋₄ alkoxy, or C₁₋₄ alkyl;

R₅ is H, F, Cl, Br, I, (C═O)Rⱼ, OH, nitro, NRⱼRₖ, cyano, phenyl, —OCH₂—Ph, C₁₋₄ alkoxy, or C₁₋₄ alkyl;

R₇ is H, F, Cl, Br, I, (C═O)Rₘ, OH, nitro, NRₗRₘ, cyano, phenyl, —OCH₂—Ph C₁₋₄ alkoxy, or C₁₋₄ alkyl;

wherein each of Rⱼ, Rₖ, Rₗ, and Rₘ is independently selected from H, C₁₋₆ alkyl, hydroxy, phenyl, benzyl, phenethyl, and C₁₋₆ alkoxy;

each of the above hydrocarbyl (including alkyl, alkoxy, phenyl, benzyl, cycloalkyl, and so on) or heterocyclic groups being independently and optionally substituted with between 1 and 3 substituents selected from C₁₋₃ alkyl, halo, hydroxy, amino, and C₁₋₃ alkoxy;

wherein n is 0, 1, or 2; where n is 2, the moiety —(CHR_{5′})ₙ₌₂— is —(CHR_{5′}—CHR_{7′})— where CHR_{5′} is between CHR_{6′} and CHR_{7′};

provided at least one of R₁, R_{2′}, R₃, R₄, R₅, R₆, and R₇ is other than H when Z is O;

and provided, where Z is O, n=1, and each of R₄, R₅, R₆, R₇, R_{2′}, R_{3′}, R_{5′}, and R_{6′} is H, (or at least 7, 8, or 9 of these 10 limitations apply) then (a) where X₂ is NH, then R₁ is (i) not methyl, pyridyl, phenyl, or benzyl, or (ii) is selected from the disclosed possibilities, but not C₁₋₂ alkyl and not a six-membered aryl or six-membered nitrogen-containing heteroaryl, or phenyl(C₁₋₂ alkylene) (alternatively, provided, where Z is O, n=1, and X₂ is NH, then at least two (or three) of R₄, R₅, R₆, R₇, R_{2′}, R_{3′}, R_{5′}, and R_{6′} is other than H); and (b) where X₂ is O, then R₁ is not methyl;

and provided, where Z is O, X₂ is NH, n=1, R₁ is methyl, each of R₄, R₆, R₇, R_{2′}, R_{3′}, R_{5′}, and R_{6′} is H (or at least 7, 8, 9, or 10 of these 11 limitations apply), then R₅ is (i) not methoxy, (ii) not methoxy, or ethoxy, (iii) not C₁₋₄ alkoxy, or (iv) not methoxy or hydroxy;

or a pharmaceutically acceptable salt, ester, or amide thereof.

According to one aspect of the invention, the invention features compounds of the following formula (Ib):

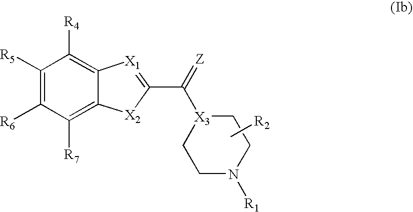

Wherein R₁ is Rₐ, RₐR_{b}—, Rₐ—O—R_{b}—, or (R_{c})(R_{d})N—R_{b}—, where Rₐ is H, C₁₋₁₀ alkyl, C₃₋₈ alkenyl, C₃₋₈ cycloalkyl, C₂₋₅ heterocyclic radical, or phenyl; where R_{b} is C₁₋₈ alkylene, C₃₋₈ alkenylene, C₃₋₈ cycloalkylene, bivalent C₃₋₈ heterocyclic radical, or phenylene; and R_{c} and R_{d} are each independently H, C₁₋₈ alkyl, C₃₋₈ alkenyl, C₃₋₈ cycloalkyl, or phenyl;

R₂ is ortho (like R_{2′} in formula (I)) or meta (like R_{3′} in formula (I)), and is methyl or H;

X₁ is CR₃;

R₃ is F, Cl, Br, R_{f}, R_{f}R_{g}—, R_{f}—O—R_{g}—, or (Rₕ)(Rᵢ)N—R_{g}—, where R_{f} is H, C₁₋₆ alkyl, C₂₋₆ alkenyl, C₃₋₆ cycloalkyl, C₂₋₅ heterocyclic radical, or phenyl; where R_{g} is C₁₋₆ alkylene, C₂₋₆ alkenylene, C₃₋₆ cycloalkylene, bivalent C₃₋₆ heterocyclic radical, or phenylene; and Rₕ and Rᵢ are each independently H, C₁₋₆ alkyl, C₂₋₆ alkenyl, C₃₋₆ cycloalkyl, or phenyl;

X₂ is NRₑ or O, provided that X₂ is NRₑ when X₁ is N; Rₑ is H or C₁₋₆ alkyl;

X₃ is N;

Z is ═O or ═S;

each of R₄ and R₆ is independently H, F, Cl, Br, I, COOH, OH, nitro, amino, cyano, C₁₋₄ alkoxy, or C₁₋₄ alkyl;

R₅ is H, F, Cl, Br, I, (C═O)Rⱼ, OH, nitro, NRⱼRₖ, cyano, —OCH₂—Ph, C₁₋₄ alkoxy, or C₁₋₄ alkyl;

R₇ is H, F, Cl, Br, I, (C═O)Rₘ, OH, nitro, NRₗRₘ, cyano, C₁₋₄ alkoxy, or C₁₋₄ alkyl;

wherein each of Rⱼ, Rₖ, Rₗ, and Rₘ is independently selected from H, C₁₋₆ alkyl, hydroxy, and C₁₋₆ alkoxy; and

each of the above hydrocarbyl or heterocyclic groups being independently and optionally substituted with between 1 and 3 substituents selected from C₁₋₃ alkyl, halo, hydroxy, amino, and C₁₋₃ alkoxy;

provided at least one of R₁, R₂, R₃, R₄, R₅, R₆, and R₇ is other than H when Z is ═O;

or a pharmaceutically acceptable salt, ester, or amide thereof.

The invention also features methods of making and using such compounds in pharmaceutical composition, packaged drugs, and in the treatment or prevention of H₄-mediated diseases and conditions, particularly those wherein it is desirable to antagonize the H₄ receptor. For example, the expression of the H₄ receptor in immune cells, including some leukocytes and mast cells, establishes it as an important target for therapeutic intervention in a range of immunological and inflammatory disorders (such as allergic, chronic, or acute inflammation). Specifically H₄ receptor ligands are expected to be useful for the treatment or prevention of various mammalian disease states. Examples include: inflammatory disorders (such as those mediated by leukocytes or mast cells), asthma, psoriasis, rheumatoid arthritis, ulcerative colitis, Crohn's disease, inflammatory bowel disease, multiple sclerosis, allergic disorders, allergic rhinitis, autoimmune disease, lymphatic disorders, atherosclerosis, and immunodeficiency disorders.

In addition, H₄ receptor ligands may be useful as adjuvants to chemotherapy. In the above methods of treatment, the invention also includes using compounds described in formula (I) and (Ib) without the provisos such as “provided at least one of R₁, R₂, R₃, R₄, R₅, R₆, and R₇ is other than H when Z is O” above in pharmaceutical compositions for treating H₄-mediated conditions, and in methods of treatment of H₄-mediated diseases. Such a compound is, for example, Example 4.

Important synthetic intermediates of the above compounds include those wherein one or more of R₄, R₅, R₆ and R₇ is Br, I, cyano, nitro, alkoxy, or —OCH₂Ph, which can be further modified to provide a wide range of substituents.

Other features and advantages of the invention will be apparent in the following detailed description, examples, and the appended claims.

### DETAILED DESCRIPTION

The invention features compounds of formulae (I) and (Ib), methods of making them, and methods of using them in the preparation of pharmaceutical compositions for the treatment or prevention of H₄-mediated diseases and conditions.

A. Terms

The following terms are defined below, and by their usage throughout the disclosure.

“Alkyl” includes straight chain and branched hydrocarbons with at least one hydrogen removed to form a radical group. Alkyl groups include methyl, ethyl, propyl, isopropyl, butyl, isobutyl, t-butyl, 1-methylpropyl, pentyl, isopentyl, sec-pentyl, hexyl, heptyl, octyl, and so on. Alkyl does not include cycloalkyl.

“Alkenyl” includes straight chain and branched hydrocarbon radicals as above with at least one carbon-carbon double bond (sp²). Alkenyls include ethenyl (or vinyl), prop-1-enyl, prop-2-enyl (or allyl), isopropenyl (or 1-methylvinyl), but-1-enyl, but-2-enyl, butadienyls, pentenyls, hexa-2,4-dienyl, and so on. Hydrocarbon radicals having a mixture of double bonds and triple bonds, such as 2-penten-4-ynyl, are grouped as alkynyls herein. Alkenyl does not include cycloalkenyl.

“Alkynyl” include straight chain and branched hydrocarbon radicals as above with at least one carbon-carbon triple bond (sp). Alkynyls include ethynyl, propynyls, butynyls, and pentynyls. Hydrocarbon radicals having a mixture of double bonds and triple bonds, such as 2-penten-4-ynyl, are grouped as alkynyls herein. Alkynyl does not include cycloalkynyl.

“Alkoxy” includes a straight chain or branched alkyl group with a terminal oxygen linking the alkyl group to the rest of the molecule. Alkoxy includes methoxy, ethoxy, propoxy, isopropoxy, butoxy, t-butoxy, pentoxy and so on. “Aminoalkyl”, “thioalkyl”, and “sulfonylalkyl” are analogous to alkoxy, replacing the terminal oxygen atom of alkoxy with, respectively, NH (or NR), S, and SO₂.

“Aryl” includes phenyl, naphthyl, biphenylyl, and so on.

“Cycloalkyl” includes cyclopropyl, cyclobutyl, cyclopentyl, cyclohexyl, cycloheptyl, cyclooctyl, and so on.

“Cycloalkenyl” includes cyclobutenyl, cyclobutadienyl, cyclopentenyl, cyclopentadienyl, cyclohexenyl, cyclohexadienyl, cyclohexatrienyl (phenyl), cycloheptenyl, and so on. “Cycloalkynyl” includes the analogous rings with one or more triple bonds.

“Heterocyclic radicals” include aromatic and nonaromatic rings having carbon atoms and at least one heteroatom (O, S, N) or heteroatom moiety (SO₂, CO, CONH, COO) in the ring. Unless otherwise indicated, a heterocyclic radical may have a valence connecting it to the rest of the molecule through a carbon atom, such as 3-furyl or 2-imidazolyl, or through a heteroatom, such as N-piperidyl or 1-pyrazolyl. Examples of heterocyclic radicals include thiazoylyl, furyl, pyranyl, isobenzofuranyl, pyrrolyl, imidazolyl, pyrazolyl, isothiazolyl, isoxazolyl, pyridyl, pyrazinyl, pyrimidinyl, pyridazinyl, indolizinyl, isoindolyl, indolyl, indazolyl, purinyl, quinolyl, furazanyl, pyrrolidinyl, pyrrolinyl, imdazolidinyl, imidazolinyl, pyrazolidinyl, pyrazolinyl, piperidyl, piperazinyl, indolinyl, and morpholinyl. For example, preferred heterocyclic radicals for Rₐ include morpholinyl, piperazinyl, pyrrolidinyl, pyridyl, cyclohexylimino, cycloheptylimino,and more preferably, piperidyl.

“Halo” includes fluoro, chloro, bromo, and iodo, and preferably fluoro or chloro.

“Patient” or “subject” includes mammals such as humans and animals (dogs, cats, horses, rats, rabbits, mice, non-human primates) in need of observation, experiment, treatment or prevention in connection with the relevant disease or condition. Preferably, the patient is a human.

“Composition” includes a product comprising the specified ingredients in the specified amounts as well as any product that results directly or indirectly from combinations of the specified ingredients in the specified amounts.

Concerning the various radicals in this disclosure and in the claims, two general remarks are made. The first remark concerns valency. As with all hydrocarbon radicals (hydrocarbyl), whether saturated, unsaturated or aromatic, and whether or not cyclic, straight chain, or branched, and also similarly with all heterocyclic radicals, each radical includes substituted radicals of that type and monovalent, bivalent, and multivalent radicals as indicated by the context of the claims. Hydrocarbyl includes alkoxy, in that the alkyl portion of an alkoxy group may be substituted. The context will indicate that the substituent is an alkylene or hydrocarbon radical with at least two hydrogen atoms removed (bivalent) or more hydrogen atoms removed (multivalent). An example of a bivalent radical linking two parts of the molecule is R_{b} in formula (I), which can link N(R_{c})(R_{d}) with the ring nitrogen atom of the rest of the molecule. Another example of a bivalent moiety is an alkylene or alkenylene.

Second, radicals or structure fragments as defined herein are understood to include substituted radicals or structure fragments. Using “alkyl” as an example, “alkyl” should be understood to include substituted alkyl having one or more substitutions, such as between 1 and 5, 1 and 3, or 2 and 4 substituents. The substituents may be the same (dihydroxy, dimethyl), similar (chlorofluoro), or different (chlorobenzyl- or aminomethyl-substituted). Examples of substituted alkyl include haloalkyl (such as fluoromethyl, chloromethyl, difluoromethyl, perchloromethyl, 2-bromoethyl, and 3-iodocyclopentyl), hydroxyalkyl, aminoalkyl, nitroalkyl, alkylalkyl, and so on. Preferred substitutions for Rₐ include methyl, methoxy, trifluoromethoxy, difluoromethoxy, fluoromethoxy, fluoromethyl, difluoromethyl, perfluoromethyl (trifluoromethyl), 1-fluoroethyl, 2-fluoroethyl, ethoxy, fluoroethoxy, fluoro, chloro, and bromo, and particularly methyl, fluoromethyl, perfluoro, trifluoromethoxy, difluoromethoxy, methoxy, and fluoro.

B. Compounds

The invention features compounds of formula (I) and (Ib). Preferred compounds include those wherein: (a) X₁ is CR₃; (b) X₃ is N; (c) X₂ is N; (d) R₁ is H, methyl, or ethyl; (e) X₂ is N and X₁ is CR₃; (f) X₂ is O and X₁ is CR₃; (9) X₂ is N and Z is O; (h) R₇ is H or Cl; (i) R₁ is methyl or ethyl; (j) R_{3′} or R_{2′} is, or both are, H; (k) R₃ is H or Cl; (I) each of R₅ and R₇ is independently selected from H, F, Cl, and Br; (m) R₃ is Cl; (n) at least one of R₅ and R₇ is F, Cl, Br, or methyl; (o) R₅, or R₇, or both is (are independently selected from) H, F, Cl, or Br; (p) R_{3′} or R_{2′} is methyl where R₁ is H; R_{3′} or R_{2′} is otherwise H; or (q) at least one of R₅ and R₇ is not H; or (r) combinations thereof.

Additional examples of preferred compounds or combinations of the above include those wherein:

(s) X₃ is N; R₃ is H or Cl; R₅ is F, Cl, Br, or methyl; and R₇ is H, F, Cl, or Br;

(t) R₃ is H or Cl; R₅ is F, Cl, Br, or methyl; and R₇ is H, F, Cl, Br, or methyl;

(u) R₂ is methyl where RI is H; R₂ is otherwise H; X₁ is CR₃; R₃ is H, F, or Cl; X₂ is NRₑ or O; Rₑ is H or C₁₋₃ alkyl; Z is ═O or =S; each of R₄ and R₆ is independently H, OH, C₁₋₄ alkyl, C₁₋₄ alkoxy, cyano, or amino; R₅ is H, F, Cl, Br, (C═O)Rⱼ, OH, amino, cyano, C₁₋₄ alkoxy, or C₁₋₄ alkyl; R₇ is H, F, Cl, Br, (C═O)Rₘ, C₁₋₄ alkyl, C₁₋₄ alkoxy, cyano, or amino; and

(v) R_{3′} and R_{2′} is methyl or H; X₁ is CR₃; R₃ is H, F, or Cl; X₂ is NRₑ or O; Rₑ is H or C₁₋₆ alkyl; Z is ═O or ═S; each of R₄ and R₆ is H; R₅ is H, F, Cl, Br, methyl, ethyl, or propyl; and R₇ is H, F, Cl, Br, or C₁₋₄ alkyl.

Examples of compounds include: (4-Methyl-piperazin-1-yl)-(5-trifluoromethyl-1H-indol-2-yl)-methanone; (7-Amino-5-methyl-1H-indol-2-yl)-(4-methyl-piperazin-1-yl)-methanone; (5-Amino-7-methyl-1H-indol-2-yl)-(4-methyl-piperazin-1-yl)-methanone; (7-Amino-5-bromo-1H-indol-2-yl)-(4-methyl-piperazin-1-yl)-methanone; (5-Amino-7-bromo-1H-indol-2-yl)-(4-methyl-piperazin-1-yl)-methanone; (5-Fluoro-7-methyl-1H-indol-2-yl)-(4-methyl-piperazin-1-yl)-methanone; (7-Fluoro-5-methyl-1H-indol-2-yl)-(4-methyl-piperazin-1-yl)-methanone; (6-Bromo-5-hydroxy-1H-indol-2-yl)-(4-methyl-piperazin-1-yl)-methanone; (5-Bromo-6-hydroxy-1H-indol-2-yl)-(4-methyl-piperazin-1-yl)-methanone; (6-Bromo-7-hydroxy-1H-indol-2-yl)-(4-methyl-piperazin-1-yl)-methanone; (4-Bromo-7-hydroxy-1H-indol-2-yl)-(4-methyl-piperazin-1-yl)-methanone; (6-Bromo-7-methyl-1H-indol-2-yl)-(4-methyl-piperazin-1-yl)-methanone; and (4-Bromo-7-methyl-1H-indol-2-yl)-(4-methyl-piperazin-1-yl)-methanone.

Additional examples of compounds include: (5,7-Dichloro-1H-indol-2-yl)-piperazin-1-yl-methanone; (5,7-Difluoro-1H-indol-2-yl)-piperazin-1-yl-methanone; (5,7-Difluoro-1H-indol-2-yl)-(3-methyl-piperazin-1-yl)-methanone; (5,6-Difluoro-1H-indol-2-yl)-(4-methyl-piperazin-1-yl)-methanone; (4,6-Difluoro-1H-indol-2-yl)-(4-methyl-piperazin-1-yl)-methanone.

Further examples of compounds include: 1-(5-Chloro-1H-indole-2-carbonyl)-4-methyl-piperazine-2-carboxylic acid methyl ester; 4-(5-Chloro-1H-indole-2-carbonyl)-1-methyl-piperazine-2-carboxylic acid methyl ester; 4-(5-Chloro-1H-indole-2-carbonyl)-1-methyl-piperazine-2-carboxylic acid amide; 1-(5-Chloro-1H-indole-2-carbonyl)-4-methyl-piperazine-2-carboxylic acid amide; 4-(5-Chloro-1H-indole-2-carbonyl)-1-methyl-piperazine-2-carboxylic acid methylamide; 1-(5-Chloro-1H-indole-2-carbonyl)-4-methyl-piperazine-2-carboxylic acid methylamide; 4-(5-Chloro-1H-indole-2-carbonyl)-1-methyl-piperazine-2-carboxylic acid dimethylamide; 1-(5-Chloro-1H-indole-2-carbonyl)-4-methyl-piperazine-2-carboxylic acid dimethylamide; (5-Chloro-1H-indol-2-yl)-(3-hydroxymethyl-4-methyl-piperazin-1-yl)-methanone; (5-Chloro-1H-indol-2-yl)-(3-methoxymethyl-4-methyl-piperazin-1-yl)-methanone; (5-Chloro-1H-indol-2-yl)-(2-methoxymethyl-4-methyl-piperazin-1-yl)-methanone; (5-Chloro-1H-indol-2-yl)-(4-methyl-3-methylaminomethyl-piperazin-1-yl)-metha none; (5-Chloro-1H-indol-2-yl)-(4-methyl-2-methylaminomethyl-piperazin-1-yl)-methanone; (5-Chloro-1H-indol-2-yl)-(3-dimethylaminomethyl-4-methyl-piperazin-1-yl)-methanone; and (5-Chloro-1H-indol-2-yl)-(2-dimethylaminomethyl-4-methyl-piperazin-1-yl)-methanone.

Examples of preferred compounds include: (5-Chloro-1H-indol-2-yl)-(4-methyl-piperazin-1-yl)-methanone; (5-Fluoro-1H-indol-2-yl)-(4-methyl-piperazin-1-yl)-methanone; (5-Bromo-1 H-indol-2-yl)-(4-methyl-piperazin-1-yl)-methanone; (5-Methyl-1H-indol-2-yl)-(4-methyl-piperazin-1-yl)-methanone; (5,7-Difluoro-1H-indol-2-yl)-(4-methyl-piperazin-1-yl)-methanone; (7-Chloro-1H-indol-2-yl)-(4-methyl-piperazin-1-yl)-methanone; (5,7-Dichloro-1H-indol-2-yl)-(4-methyl-piperazin-1-yl)-methanone; and (3,5-Dichloro-1H-indol-2-yl)-(4-methyl-piperazin-1-yl)-methanone. More preferred compounds in this group include (5-Chloro-1H-indol-2-yl)-(4-methyl-piperazin-1-yl)-methanone; (5-Bromo-1H-indol-2-yl)-(4-methyl-piperazin-1-yl)-methanone; (5-Methyl-1H-indol-2-yl)-(4-methyl-piperazin-1-yl)-methanone; (5,7-Difluoro-1H-indol-2-yl)-(4-methyl-piperazin-1-yl)-methanone; (5-Fluoro-1H-indol-2-yl)-(4-methyl-piperazin-1-yl)- methanone; (7-Amino-1H-indol-2-yl)-(4-methyl-piperazin-1-yl)-methanone; (7-Methyl-1H-indol-2-yl)-(4-methyl-piperazin-1-yl)-methanone; and (5,7-Dichloro-1H-indol-2-yl)-(4-methyl-piperazin-1-yl)-methanone.

Further examples of preferred compounds include (6-Chloro-1H-indol-2-yl)-(4-methyl-piperazin-1-yl)-methanone; (1 H-Indol-2-yl)-(3-methyl-piperazin-1-yl)-methanone; (7-Bromo-1H-indol-2-yl)-(4-methyl-piperazin-1-yl)-methanone; (5-Bromo-benzofuran-2-yl)-(4-methyl-piperazin-1-yl)-methanone; and (1H-Indol-2-yl)-(4-methyl-piperazin-1-yl)-methanethione.

The most preferred compound is (5-Chloro-1H-indol-2-yl)-(4-methyl-piperazin-1-yl)-methanone.

The disclosed compounds can be prepared according to the next section.

C. Synthesis

The disclosed compounds may be made by combinatorial or traditional organic synthetic methods, as outlined below in Schemes 1-12 and Chemical Examples 1-86, or by analogous reactions.

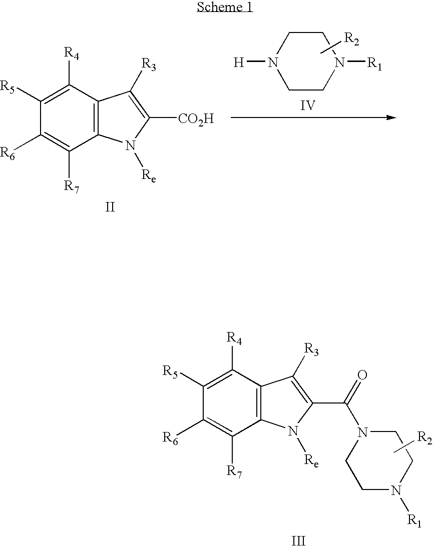

Compounds of formula III may be prepared from the compounds of formula II using conventional methods of amide bond formation. For example the carboxyl group of compound II may be activated as an active ester, acid chloride, anhydride, mixed anhydride, carbonic mixed anhydride or the like and treated with an amine containing group to give a compound of formula III. For example the compound of formula II may be converted to the corresponding active ester upon treatment with 1-hydroxybenzotriazole in the presence of a carbodiimide for example dicyclohexylcarbodiimide or 1-ethyl-3-(3′-dimethyl-aminopropyl)-carbodiimide hydrochloride in the presence of a base such as triethylamine or N,N-diisopropylethylamine to give a compound of formula III. In a preferred embodiment the compound of formula II is treated with O-(7-azabenzotriazol-1-yl)-N,N,N′,N′-tetramethyluroniumhexafluoro-phosphate, (HATU) and 1-hydroxy-7-azabenzotriazole, (HOAT) and N,N-diisopropylethylamine in a solvent, for example DMF, THF or the like, together with an amine component IV to give a compound of formula II. In an additional preferred embodiment a compound of formula II may be treated with carbonyldiimidazole (CDI) in a solvent, for example THF, DMF, dichloromethane or the like, followed by an amine component IV to give a compound of formula III.

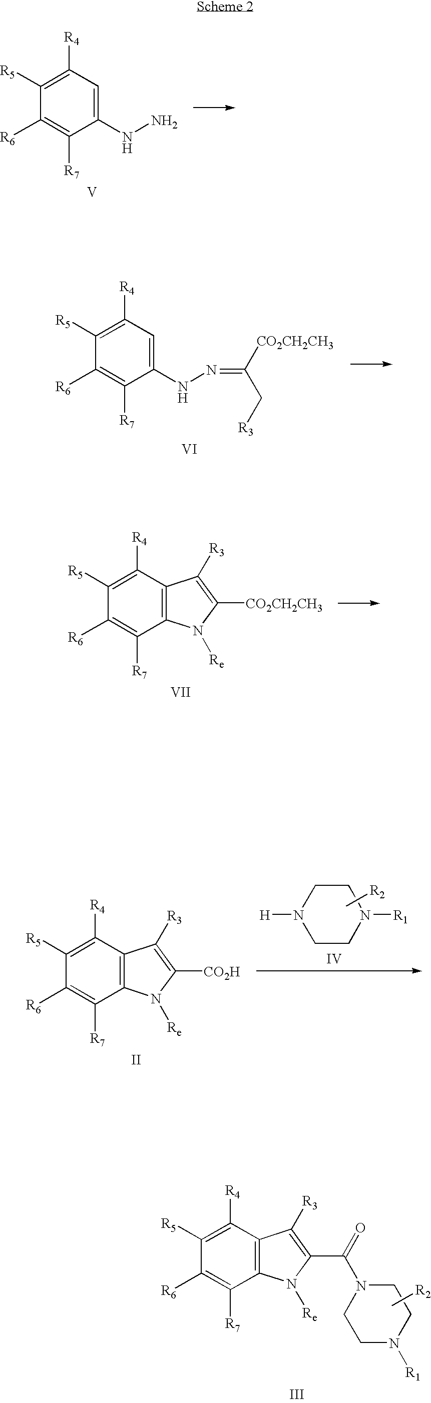

Compounds of formula III may be prepared according to the Fischer-Indole synthesis, which involves the condensation of a phenylhydrazine with an aldehyde or ketone to give an intermediate hydrazone. Thus a compound of formula V may be condensed with ethylpyruvate, usually in the presence of an acid catalyst, for example sulfuric acid to afford a hydrazone of formula VI. Compounds of formula VI may be converted into indoles of formula VII upon treatment with a protic or Lewis acid, if required at elevated temperature, to effect cyclisation. Examples of acids include; polyphosphoric acid, para-toluenesulfonic acid, pyridine hydrochloride, zinc chloride, phosphorus trichloride, polyphosphoric acid trimethylsilyl ester and acetic acid. Compound VI may also be converted to compound VII under thermal conditions by heating a compound of formula VI in a solvent, for example ethylene glycol, tetralin, or the like at elevated temperature, for example at about 150 to 250° C. It will be recognized by one skilled in the art that cyclization of compounds of formula VI to compounds of formula VII can give rise to isomers when compounds of formula V contain substituents. It will be further recognized that the conditions to effect cyclization may be different for different compounds of formula VI.

In a further embodiment, compounds of formula VII may be prepared by condensing an appropriately substituted 2-nitrotoluene with an oxalate di-ester in the presence of a base followed by reduction of the intermediate to afford a compound of formula VII. In a preferred embodiment, a 2-nitrotoluene is condensed with ethylpyruvate in the presence of a base such as sodium methoxide, sodium butoxide, or sodium ethoxide in a solvent such as ethanol, methanol, or butanol. For example, a solution of 2-nitrotoluene in ethanol is heated with ethylpyruvate in the presence of sodium ethoxide at reflux temperature. The condensation product may be converted to a compound of formula VII using a reducing agent, preferably zinc in aqueous acetic acid. Compounds of formula VII may be converted to compounds of formula II using standard methods for ester hydrolysis, for example upon treatment with aqueous acid or base, if necessary at elevated temperature. In a preferred embodiment hydrolysis may be effected upon treating a compound of formula VII with a solution of lithium hydroxide in an alcoholic solvent, preferably ethanol. Compounds of formula II may be converted to compounds of formula III according to the procedures described previously.

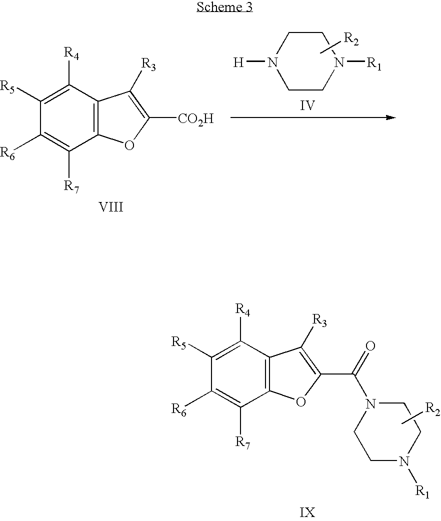

Compounds of formula IX may be prepared from the compounds of formula VIII using conventional methods of amide bond formation as described for the preparation of compounds of formula III from compounds of formula II by condensing the appropriate carboxylic acid of formula VIII with an amine component IV.

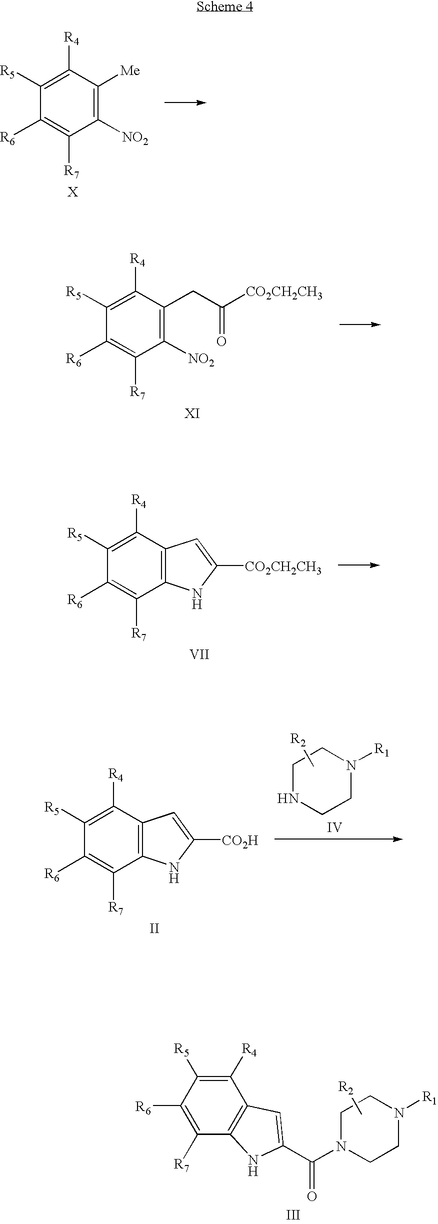

Compounds of formula III may also be prepared as depicted in Scheme 4. Treatment of an optionally substituted 2-nitrotoluene (formula X) with an oxalate, such as diethyl oxalate, in the presence of a base affords a 2-keto ester of formula XI. Typical bases used to effect this transformation include potassium ethoxide, sodium hydride, and lithium t-butoxide. Reduction of the nitro group of a compound of formula XI to the corresponding aniline is accompanied by cyclization to the indole 2-carboxylate, a compound of formula VII. Typical reducants for this transformation include hydrogen over palladium, tin(II) chloride, and sulfur. Compounds of formula VII may be converted to compounds of formula II using standard methods for ester hydrolysis, for example upon treatment with aqueous acid or base, if necessary at elevated temperature. In a preferred embodiment hydrolysis may be effected upon treating a compound of formula VII with a solution of lithium hydroxide in THF. Conversion to the target compounds III is effected as described in Scheme 2.

Formulae XII and XIII do not exist in this disclosure.

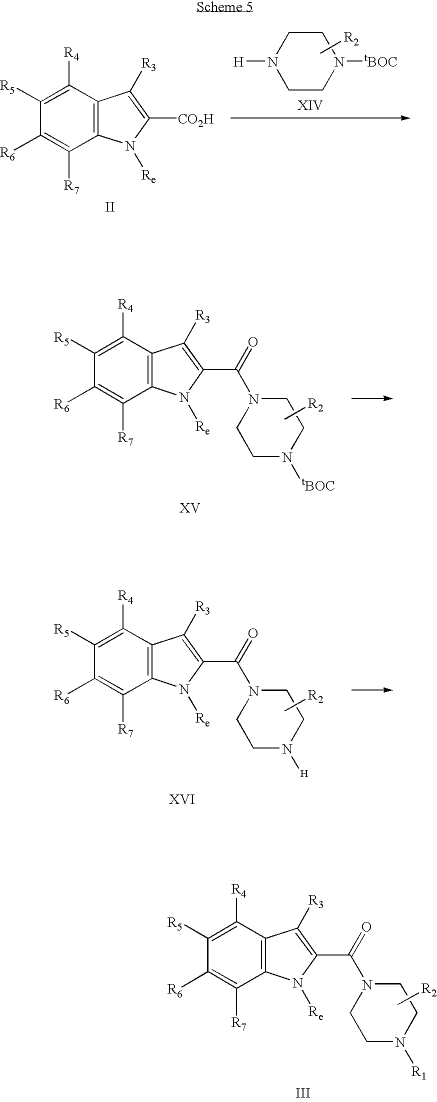

Compounds of formula III may be also be prepared from compounds of formula II by condensing a piperazine-1-carboxylic acid tert-butyl ester of formula XIV with a compound of formula II using conventional methods of amide bond formation as described for the preparation of compounds of formula III from compounds of formula II. In a preferred embodiment a compound of formula II is treated with carbonyldiimidazole (CDI) in a solvent, for example THF, DMF, dichloromethane or the like, followed a piperazine-1-carboxylic acid tert-butyl ester of formula XIV to afford a compound of formula XV. Compound XV may be converted to a compound of formula XVI upon treatment with an acid, for example trifluoroacetic acid or hydrochloric acid in a solvent, for example dichloromethane, THF, dioxane or the like. In a preferred embodiment the acid is trifluoroacetic acid and the solvent dichloromethane. A compound of formula III may be obtained from a compound of formula XVI upon treatment with an alkylating agent in the presence of a base. Suitable alkylating agents include, alkylbromides, alkylchlorides, alkyliodides, alkylmesylates, and alkyltosylates. This transformation is effected in the presence of a base, for example potassium carbonate, sodium hydroxide, triethylamine and the like, in a solvent, for example ethanol, methanol, acetone, dichloromethane, DMF, THF and the like. Preferred conditions use potassium carbonate in acetone. The reaction may be carried out at elevated temperature, preferably at about 50° C.

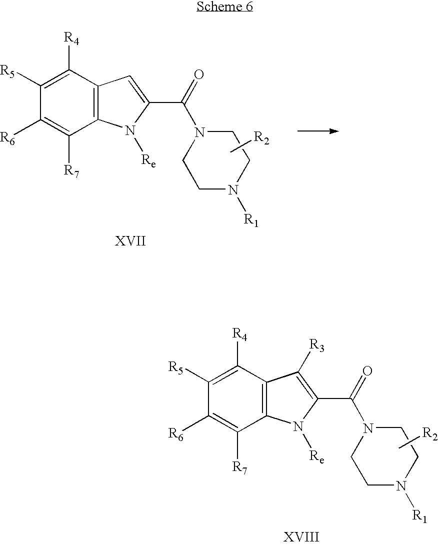

Compounds of formula XVIII may be prepared from compounds of formula XVII according to known methods for the functionalization of the indole nucleus at C-3. Such methods include, but are not limited to; halogenation, for example treatment with a halogen source in a solvent, for example upon treatment with bromine in acetic acid, N-chlorosuccinamide, N-bromosuccinamide, N-iodosuccinamide in dichloromethane, carbontetrachloride, chloroform or the like; formylation, for example by heating a DMF solution of a compound of formula XVII with phosphorus oxychloride (Vilsmeier-Haack conditions); aminoalkylation, for example by treating a compound of formula XVII with a mixture of am amine and a source of formaldehyde (Mannich conditions). One skilled in the art will recognize that not all reactions of indoles with electrophiles will lead to substitution at C-3 alone and that additional substitution may also take place and that mixtures of products may be obtained. It may be further recognized that the products of the substitution reactions (3-substituted indoles) may be used for further transformations.

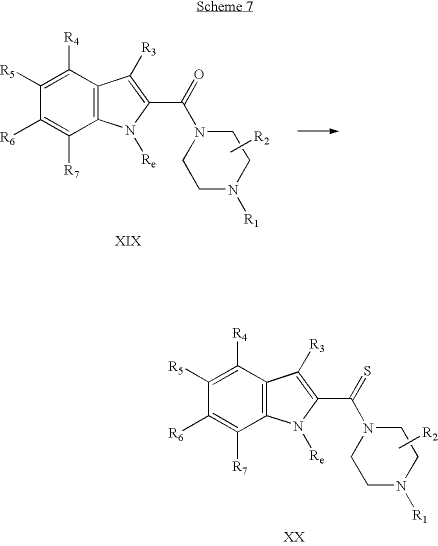

A compound of formula XX may be obtained from a compound of formula XIX upon treatment with 2,4-bis(4-methoxyphenyl)-1,3,2,4-dithiadiphosphetane-2,4-disulfide (also known as Lawesson's reagent) in a solvent for example ether, THF or dioxane. In a preferred embodiment the compound of formula XIX is treated with Lawessons's reagent in THF at ambient temperature to give a compound of formula XX.

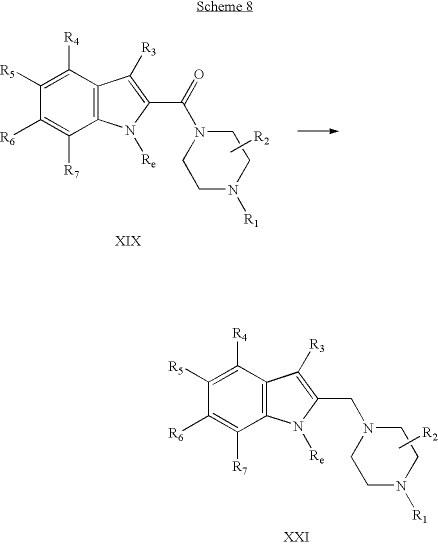

A compound of formula XXI may be obtained from a compound of formula XIX using conventional methods for amide bond reduction. For example using lithium aluminum hydride in THF, magnesium aluminum hydride in THF, lithium trimethoxyaluminum hydride, sodium bis(2-methoxyethoxy)-aluminum hydride, alane in THF and borane or borane-dimethyl sulfide complex in THF. A preferred method is the use of lithium aluminum hydride in a solvent, for example THF, dioxane, ether or the like at from 25° C. to the boiling point of the selected solvent. In a more preferred embodiment the reducing agent is lithium aluminum hydride in THF at reflux temperature. As shown in the scheme below, compounds of formula XI may be prepared by utilizing a Phillips-type reaction that involves the condensation of an ortho-arylene diamine with a carboxcylic acid or the like, to generate the benzimidazole core. Accordingly, a compound of formula XXII may be

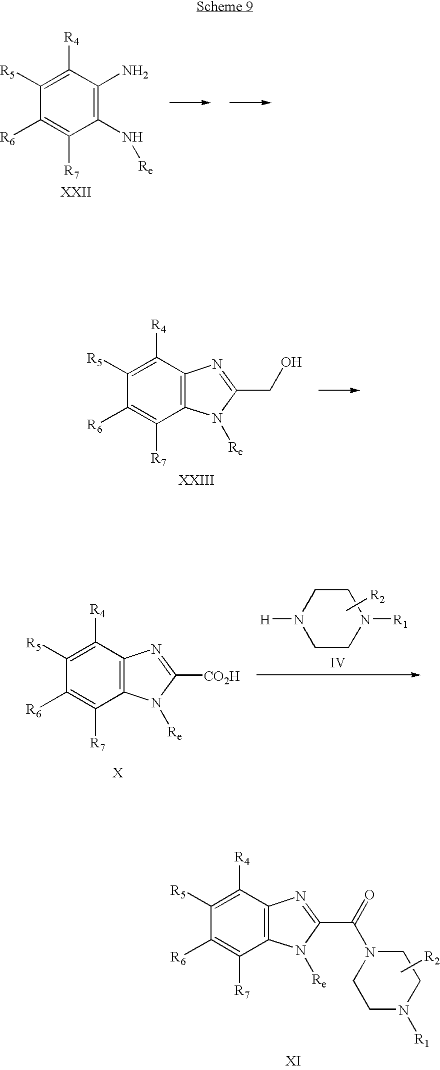

condensed with glycolic acid and typically with an acid catalyst, for example hydrochloric acid, to afford compounds of formula XXIII. It will be recognized by one skilled in the art that the condensation of compounds of formula XXII to compounds of formula XXIII can give rise to isomers when compounds of formula XXII contain substituents. Compounds of formula XXIII may be oxidized with a suitable oxidizing agent to give compounds of formula X. Oxidants may include potassium permanganate, chromium trioxide, sodium hypochlorite, dimethyl sulfoxide with oxalyl chloride, manganese dioxide or any combination thereof. Compounds of formula X may be converted to compounds of formula XI according to the procedures described previously for compounds of formula II by condensing the appropriate carboxylic acid of formula X with an amine component IV.

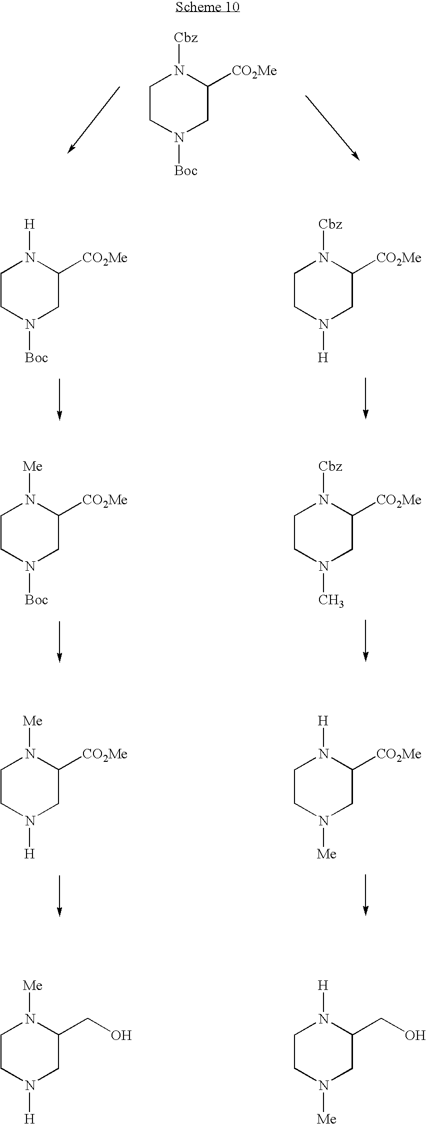

Scheme 10 illustrates methods of making substituted proximal and distal regioisomers. Analogous methods may be used with rings of other than 6 members, such as 5- or 7-membered rings. Further modifications may be made to change the hydroxymethyl and the methyl ester substituents using methods well known to those skilled in the art, including, but not limited to, those methods detailed in Schemes 11 and 12. Piperazine-1,2,4-tricarboxylic acid 1-benzyl ester 4-tert-butyl ester 2-methyl ester can be prepared according to the procedure of Bigge et al. (Tetrahedron Lett. 1989, 30:5193-5196). Selective deprotection of either the CBz or the BOC group can be accomplished using standard methods. For example, selective removal of the CBz group of piperazine-1,2,4-tricarboxylic acid 1-benzyl ester 4-tert-butyl ester 2-methyl ester can be accomplished upon treatment with, but not limited to, H₂ and Pd/C or ammonium formate and Pd/C in solvents such as ethanol or ethyl acetate or the like, to give piperazine-1,3-dicarboxylic acid 1-tert-butyl ester 3-methyl ester. Conversion of piperazine-1,3-dicarboxylic acid 1-tert-butyl ester 3-methyl ester to 4-methyl-piperazine-1,3-dicarboxylic acid 1-tert-butyl ester 3-methyl ester can be accomplished using standard conditions for reductive amination. These include, but are not limited to, treatment with paraformaldehyde in the presence of a reducing agent such as sodium borohydride, sodium cyanoborohydride or sodium triacetoxyborohydride or the like, in a solvent such as tetrahydrofuran, methanol, ethanol, 1,2-dichloroethane, trifluoroethanol, or the like. One skilled in the art will recognize that addition of acid to decrease the pH of the reaction mixture to a pH of less than about 7 may be necessary to effect reaction, wherein the acid is added as needed and is such as acetic acid, hydrochloric acid, and the like. Preferred reducing agents are sodium cyanoborohydride or sodium triacetoxyboro-hydride. Removal the the BOC group can be accomplished upon treatment with an acid, for example trifluoroacetic acid or hydrochloric acid in a solvent, for example dichloromethane, THF, dioxane or the like to give 1-methyl-piperazine-2-carboxylic acid methyl ester. Reduction of the methyl ester can be accomplished using standard conditions including, but not limited to, treatment with reducing agents such as lithium aluminum hydride or diisobutylaluminum hydride or the like, in solvents such as THF or diethyl ether or the like to afford (1-methyl-piperazin-2-yl)-methanol.

Alternatively, selective removal of the BOC group of piperazine-1,2,4-tricarboxylic acid 1-benzyl ester 4-tert-butyl ester 2-methyl ester can be accomplished upon treatment with an acid, for example trifluoroacetic acid or hydrochloric acid in a solvent, for example dichloromethane, THF, dioxane or the like to give piperazine-1,2-dicarboxylic acid 1-benzyl ester 2-methyl ester. Conversion of piperazine-1,2-dicarboxylic acid 1-benzyl ester 2-methyl ester to 4-methyl-piperazine-1,2-dicarboxylic acid 1-benzyl ester 2-methyl ester can be accomplished using standard conditions for reductive amination. These include, but are not limited to, treatment with paraformaldehyde in the presence of a reducing agent such as sodium borohydride, sodium cyanoborohydride or sodium triacetoxyborohydride, or the like, in a solvent such as tetrahydrofuran, methanol, ethanol, 1,2-dichloroethane, trifluoroethanol, or the like. One skilled in the art will recognize that addition of acid to decrease the pH of the reaction mixture to a pH of less than about 7 may be necessary to effect reaction, wherein the acid is added as needed and is such as acetic acid, hydrochloric acid, or the like. Preferred reducing agents are sodium cyanoborohydride or sodium triacetoxyborohydride. Removal of the CBz group of 4-methyl-piperazine-1,2-dicarboxylic acid 1-benzyl ester 2-methyl ester can be accomplished upon treatment with, but not limited to, H₂ and Pd/C or ammonium formate and Pd/C in sovents such as ethanol or ethyl acetate or the like, to give 4-methyl-piperazine-2-carboxylic acid methyl ester. Reduction of the methyl ester can be accomplished using standard conditions including, but not limited, to treatment with reducing agents such as lithium aluminum hydride or diisobutylaluminum hydride or the like, in solvents such as THF or diethyl ether or the like, to afford (4-methyl-piperazin-2-yl)-methanol.

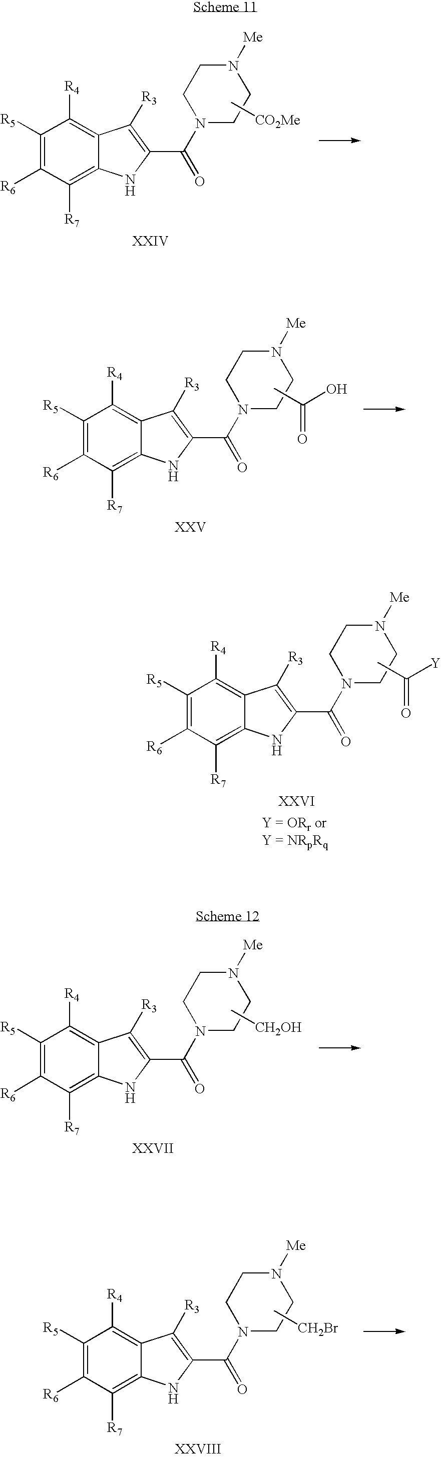

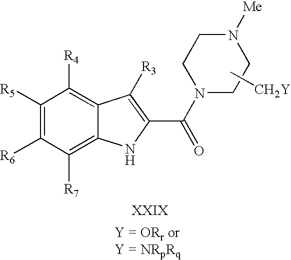

Compounds of formulas XXIV and XXVII may be prepared from compounds of formula II using conventional methods of amide bond formation, as described for the preparation of compounds of formula III from compounds of formula II, by condensing the appropriate carboxylic acid of formula II with an amine component such as those described in Scheme 10. Schemes 11 and 12 illustrate non-limiting methods for providing the substituted rings, such as the substituted piperazines shown in compounds XXVI and XXIX. For Scheme 11, hydrolysis of the ester can be accomplished using standard methods for ester hydrolysis, for example upon treatment with aqueous acid or base, if necessary at elevated temperature. Compounds of formula XXVI where Y is nitrogen can be prepared using conventional methods of amide bond formation, as described for the preparation of compounds of formula III from compounds of formula II, by condensing the appropriate carboxylic acid of formula XXV with a suitable amine component. Compounds of formula XXVI where Y is oxygen can be prepared using conventional methods of ester formation such as, but not limited to, conversion to the acid chloride using reagents such as oxalyl chloride, or the like, followed by treatment with an appropriate alcohol. For Scheme 12, compounds of formula XXVIII can be prepared from compounds of formula XXVII using conventional methods such as, but not limited to, treatment with triphenylphosphine and carbon tetrabromide, thionyl bromide or HBr. Compounds of formula XXVIII may be treated with alcohols or amines to afford compounds of formula XXIX where Y is oxygen or nitrogen respectively, possibly in the presense of a suitable base such as, but not limited to, cesium carbonate or triethylamine.

D. Uses

According to the invention, the disclosed compounds and compositions are useful for the amelioration of symptoms associated with, the treatment of, and the prevention of, the following conditions and diseases: inflammatory disorders, asthma, atherosclerosis, psoriasis, rheumatoid arthritis, ulcerative colitis, Crohn's disease, inflammatory bowel disease, multiple sclerosis, allergic disorders, allergic rhinitis, dermatological disorders, autoimmune disease, lymphatic disorders, and immunodeficiency disorders. The disclosed compounds may also be useful as adjuvants in chemotherapy or in the treatment of itchy skin. The invention also features pharmaceutical compositions that include, without limitation, one or more of the disclosed compounds, and pharmaceutically acceptable carrier or excipient.

Aspects of the invention include (a) a pharmaceutical composition comprising a compound of formula (I) or (Ib), or one or more preferred compounds as described herein, and a pharmaceutically acceptable carrier; (b) a packaged drug comprising (1) a pharmaceutical composition comprising a compound of claim 1, 2, or 3 and a pharmaceutically acceptable carrier, and (2) instructions for the administration of said composition for the treatment or prevention of an H₄-mediated disease or condition.

The invention also provides a method for treating an H₄-mediated condition in a patient, said method comprising administering to the patient a pharmaceutically effective amount of a composition comprising a compound of formula (I) or (Ib) or other disclosed or preferred compounds. For example, the invention features a method for treating an H₄ mediated condition in a patient, said method comprising administering to the patient a pharmaceutically effective H₄-antagonizing amount of a composition comprising a compound of formula (I) or (Ib) or other disclosed or preferred compounds.

The effect of an antagonist may also be produced by an inverse agonist. Inverse agonism describes the property of a compound to actively turn off a receptor that displays constitutive activity. Constitutive activity can be identified in cells that have been forced to over-express the human H₄ receptor. Constitutive activity can be measured by examining cAMP levels or by measuring a reporter gene sensitive to cAMP levels after a treatment with a cAMP-stimulating agent such as forskolin. Cells that over-express H₄ receptors will display lower cAMP levels after forskolin treatment than non-expressing cells. Compounds that behave as H₄ agonists will dose-dependently lower forskolin-stimulated cAMP levels in H₄-expressing cells. Compounds that behave as inverse H₄ agonists will dose-dependently stimulate cAMP levels in H₄-expressing cells. Compounds that behave as H₄ antagonists will block either H₄ agonist-induced inhibition of cAMP or inverse H₄ agonist-induced increases in cAMP.

Further embodiments of the invention include disclosed compounds that are inhibitors of a mammalian histamine H₄ receptor function, inhibitors of inflammation or inflammatory responses in vivo or in vitro, modulators of the expression of a mammalian histamine H₄ receptor protein, inhibitors of polymorphonuclear leukocyte activation in vivo or in vitro, or combinations of the above, and corresponding methods of treatment, prophylaxis, and diagnosis comprising the use of a disclosed compound.

1. Dosages

Those skilled in the art will be able to determine, according to known methods, the appropriate dosage for a patient, taking into account factors such as age, weight, general health, the type of symptoms requiring treatment, and the presence of other medications. In general, an effective amount will be between 0.01 and 1000 mg/kg per day, preferably between 0.5 and 300 mg/kg body weight, and daily dosages will be between 10 and 5000 mg for an adult subject of normal weight. Capsules, tablets or other formulations (such as liquids and film-coated tablets) may be of between 0.5 and 200 mg, such as 1, 3, 5, 10, 15, 25, 35, 50 mg, 60 mg, and 100 mg and can be administered according to the disclosed methods.

2. Formulations

Dosage unit forms include tablets, capsules, pills, powders, granules, aqueous and nonaqueous oral solutions and suspensions, and parenteral solutions packaged in containers adapted for subdivision into individual doses. Dosage unit forms can also be adapted for various methods of administration, including controlled release formulations, such as subcutaneous implants. Administration methods include oral, rectal, parenteral (intravenous, intramuscular, subcutaneous), intracisternal, intravaginal, intraperitoneal, intravesical, local (drops, powders, ointments, gels or cream), and by inhalation (a buccal or nasal spray).

Parenteral formulations include pharmaceutically acceptable aqueous or nonaqueous solutions, dispersion, suspensions, emulsions, and sterile powders for the preparation thereof. Examples of carriers include water, ethanol, polyols (propylene glycol, polyethylene glycol), vegetable oils, and injectable organic esters such as ethyl oleate. Fluidity can be maintained by the use of a coating such as lecithin, a surfactant, or maintaining appropriate particle size. Carriers for solid dosage forms include (a) fillers or extenders, (b) binders, (c) humectants, (d) disintegrating agents, (e) solution retarders, (f) absorption accelerators, (g) adsorbants, (h) lubricants, (i) buffering agents, and (j) propellants.

Compositions may also contain adjuvants such as preserving, wetting, emulsifying, and dispensing agents; antimicrobial agents such as parabens, chlorobutanol, phenol, and sorbic acid; isotonic agents such as a sugar or sodium chloride; absorption-prolonging agents such as aluminum monostearate and gelatin; and absorption-enhancing agents.

3. Related Compounds

The invention provides the disclosed compounds and closely related, pharmaceutically acceptable forms of the disclosed compounds, such as salts, esters, amides, hydrates or solvated forms thereof; masked or protected forms; and racemic mixtures, or enantiomerically or optically pure forms.

Pharmaceutically acceptable salts, esters, and amides include carboxylate salts (e.g., C₁₋₈ alkyl, cycloalkyl, aryl, heteroaryl, or non-aromatic heterocyclic) amino acid addition salts, esters, and amides that are within a reasonable benefit/risk ratio, pharmacologically effective and suitable for contact with the tissues of patients without undue toxicity, irritation, or allergic response. Representative salts include hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, and laurylsulfonate. These may include alkali metal and alkali earth cations such as sodium, potassium, calcium, and magnesium, as well as non-toxic ammonium, quaternary ammonium, and amine cations such as tetramethyl ammonium, methylamine, trimethylamine, and ethylamine. See example, S. M. Berge, et al., “Pharmaceutical Salts,” J. Pharm. Sci., 1977, 66:1-19, which is incorporated herein by reference. Representative pharmaceutically acceptable amides of the invention include those derived from ammonia, primary C₁₋₆ alkyl amines and secondary di (C₁₋₆ alkyl) amines. Secondary amines include 5- or 6-membered heterocyclic or heteroaromatic ring moieties containing at least one nitrogen atom and optionally between 1 and 2 additional heteroatoms. Preferred amides are derived from ammonia, C₁₋₃ alkyl primary amines, and di (C₁₋₂ alkyl)amines. Representative pharmaceutically acceptable esters of the invention include C₁₋₇ alkyl, C₅₋₇ cycloalkyl, phenyl, and phenyl(C₁₋₆)alkyl esters. Preferred esters include methyl esters.

The invention also includes disclosed compounds having one or more functional groups (e.g., hydroxyl, amino, or carboxyl) masked by a protecting group. Some of these masked or protected compounds are pharmaceutically acceptable; others will be useful as intermediates. Synthetic intermediates and processes disclosed herein, and minor modifications thereof, are also within the scope of the invention.

Hydroxyl Protecting Groups

Protection for the hydroxyl group includes methyl ethers, substituted methyl ethers, substituted ethyl ethers, substitute benzyl ethers, and silyl ethers.

Substituted Methyl Ethers

Examples of substituted methyl ethers include methyoxymethyl, methylthiomethyl, t-butylthiomethyl, (phenyidimethylsilyl)methoxymethyl, benzyloxymethyl, p-methoxybenzyloxymethyl, (4-methoxyphenoxy)methyl, guaiacolmethyl, t-butoxymethyl, 4-pentenyloxymethyl, siloxymethyl, 2-methoxyethoxymethyl, 2,2,2-trichloroethoxymethyl, bis(2-chloroethoxy)methyl, 2-(trimethylsilyl)ethoxymethyl, tetrahydropyranyl, 3-bromotetrahydropyranyl, tetrahydrothiopyranyl, 1-methoxycyclohexyl, 4-methoxytetrahydropyranyl, 4-methoxytetrahydrothiopyranyl, 4-methoxytetrahydrothiopyranyl S,S-dioxido, 1-[(2-chloro-4-methyl)phenyl]-4-methoxypiperidin4-yl, 1,4-dioxan-2-yl, tetrahydrofuranyl, tetrahydrothiofuranyl and 2,3,3a,4,5,6,7,7a-octahydro-7,8,8-trimethyl-4,7-methanobenzofuran-2-yl.

Substituted Ethvl Ethers

Examples of substituted ethyl ethers include 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, and benzyl.

Substituted Benzyl Ethers

Examples of substituted benzyl ethers include p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2- and 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxy)phenyldiphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(Imidazol-1-ylmethyl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, and benzisothiazolyl S,S-dioxido.

Silyl Ethers

Examples of silyl ethers include trimethylsilyl, triethylsilyl, triisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, and t-butylmethoxyphenylsilyl.

Esters

In addition to ethers, a hydroxyl group may be protected as an ester. Examples of esters include formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, p-P-phenylacetate, 3-phenylpropionate, 4-oxopentanoate(levulinate), 4,4-(ethylenedithio)pentanoate, pivaloate, adamantoate, crotonate, 4-methoxycrotonate, benzoate, p-phenylbenzoate, 2,4,6-trimethylbenzoate(mesitoate)

Carbonates

Examples of carbonates include methyl, 9-fluorenylmethyl, ethyl, 2,2,2-trichloroethyl, 2-(trimethylsilyl)ethyl, 2-(phenylsulfonyl)ethyl, 2-(triphenylphosphonio)ethyl, isobutyl, vinyl, allyl, p-nitrophenyl, benzyl, p-methoxybenzyl, 3,4-dimethoxybenzyl, o-nitrobenzyl, p-nitrobenzyl, S-benzyl thiocarbonate, 4-ethoxy-1-naphthyl, and methyl dithiocarbonate.

Assisted Cleavage

Examples of assisted cleavage include 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate, 4-(methylthiomethoxy)butyrate, and 2-(methylthiomethoxymethyl)benzoate.

Miscellaneous Esters

Examples of miscellaneous esters include 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate(tigloate), o-(methoxycarbonyl)benzoate, p-P-benzoate, α-naphthoate, nitrate, alkyl N,N,N′,N′-tetramethylphosphorodiamidate, N-phenylcarbamate, borate, dimethylphosphinothioyl, and 2,4-dinitrophenylsulfenate

Sulfonates

Examples of sulfonates include sulfate, methanesulfonate(mesylate), benzylsulfonate, and tosylate.

Protection for 1,2- and 1,3-Diols

Cyclic Acetals and Ketals

Examples of cyclic acetals and ketals include methylene, ethylidene, 1-t-butylethylidene, 1-phenylethylidene, (4-methoxyphenyl)ethylidene, 2,2,2-trichloroethylidene, acetonide (isopropylidene), cyclopentylidene, cyclohexylidene, cycloheptylidene, benzylidene, p-methoxybenzylidene, 2,4-dimethoxybenzylidene, 3,4-dimethoxybenzylidene, and 2-nitrobenzylidene.

Cyclic Ortho Esters

Examples of cyclic ortho esters include methoxymethylene, ethoxymethylene, dimethoxymethylene, 1-methoxyethylidene, 1-ethoxyethylidine, 1,2-dimethoxyethylidene, α-methoxybenzylidene, 1-(N,N-dimethylamino)ethylidene derivative, a-(N,N-dimethylamino)benzylidene derivative, and 2-oxacyclopentylidene.

Silyl Derivatives

Examples of silyl derivatives include di-t-butylsilylene group, and 1,3-(1,1,3,3-tetraisopropyldisiloxanylidene) derivative.

Amino Protecting Groups

Protection for the amino group includes carbamates, amides, and special —NH protective groups.

Examples of carbamates include methyl and ethyl carbamates, substituted ethyl carbamates, assisted cleavage carbamates, photolytic cleavage carbamates, urea-type derivatives, and miscellaneous carbamates.

Carbamates

Examples of methyl and ethyl carbamates include methyl and ethyl, 9-fluorenylmethyl, 9-(2-sulfo)fluorenylmethyl, 9-(2,7-dibromo)fluorenylmethyl, 2,7-di-t-butyl-[9-(10,10-dioxo-10,10,10,10-tetrahydrothioxanthyl)]methyl, and 4-methoxyphenacyl.

Substituted Ethyl

Examples of substituted ethyl carbamates include 2,2,2-trichloroethyl, 2-trimethylsilylethyl, 2-phenylethyl, 1-(1-adamantyl)-1-methylethyl, 1,1-dimethyl-2-haloethyl, 1,1-dimethyl-2,2-dibromoethyl, 1,1-dimethyl-2,2,2-trichloroethyl, 1-methyl-1-(4-biphenylyl)ethyl, 1-(3,5-di-t-butylphenyl)-1-methylethyl, 2-(2′- and 4′-pyridyl)ethyl, 2-(N,N-dicyclohexylcarboxamido)ethyl, t-butyl, 1-adamantyl, vinyl, allyl, 1-isopropylallyl, cinnamyl, 4-nitrocinnamyl, 8-quinolyl, N-hydroxypiperidinyl, alkyldithio, benzyl, p-methoxybenzyl, p-nitrobenzyl, p-bromobenzyl, p-chlorobenzyl, 2,4-dichlorobenzyl, 4-methylsulfinylbenzyl, 9-anthrylmethyl and diphenylmethyl.

Assisted Cleavage

Examples of assisted cleavage include 2-methylthioethyl, 2-methylsulfonylethyl, 2-(p-toluenesulfonyl)ethyl, [2-(1,3-dithianyl)]methyl, 4-methylthiophenyl, 2,4-dimethylthiophenyl, 2-phosphonioethyl, 2-triphenylphosphonioisopropyl, 1,1-dimethyl-2-cyanoethyl, m-chloro-p-acyloxybenzyl, p-(dihydroxyboryl)benzyl, 5-benzisoxazolylmethyl, and 2-(trifluoromethyl)-6-chromonylmethyl.

Photolytic Cleavage

Examples of photolytic cleavage include m-nitrophenyl, 3,5-dimethoxybenzyl, o-nitrobenzyl, 3,4-dimethoxy-6-nitrobenzyl, and phenyl(o-nitrophenyl)methyl.

Urea-Type Derivatives

Examples of urea-type derivatives include phenothiazinyl-(10)-carbonyl derivative, N′-p-toluenesulfonylaminocarbonyl, and N′-phenylaminothiocarbonyl.

Miscellaneous Carbamates

Examples of miscellaneous carbamates include t-amyl, S-benzyl thiocarbamate, p-cyanobenzyl, cyclobutyl, cyclohexyl, cyclopentyl, cyclopropylmethyl, p-decyloxybenzyl, diisopropylmethyl, 2,2-dimethoxycarbonylvinyl, o-(N,N-dimethylcarboxamido)benzyl, 1,1-dimethyl-3-(N,N-dimethylcarboxamido)propyl, 1,1-dimethylpropynyl, di(2-pyridyl)methyl, 2-furanylmethyl, 2-iodoethyl, isobornyl, isobutyl, isonicotinyl, p-(p′-methoxyphenylazo)benzyl, 1-methylcyclobutyl, 1-methylcyclohexyl, 1-methyl-1-cyclopropylmethyl, 1-methyl-1-(3,5-dimethoxyphenyl)ethyl, 1-methyl-1-(p-phenylazophenyl)ethyl, 1-methyl-1-phenylethyl, 1-methyl-1-(4-pyridyl)ethyl, phenyl, p-(phenylazo)benzyl, 2,4,6-tri-t-butylphenyl, 4-(trimethylammonium)benzyl, and 2,4,6-trimethylbenzyl.

Examples of Amides Include:

Amides

N-formyl, N-acetyl, N-chloroacetyl, N-trichloroacetyl, N-trifluoroacetyl, N-phenylacetyl, N-3-phenylpropionyl, N-picolinoyl, N-3-pyridylcarboxamide, N-benzoylphenylalanyl derivative, N-benzoyl, N-p-phenylbenzoyl.

Assisted Cleavaqe

N-o-nitrophenylacetyl, N-o-nitrophenoxyacetyl, N-acetoacetyl, (N′-dithiobenzyloxycarbonylamino)acetyl, N-3-(p-hydroxyphenyl)propionyl, N-3-(o-nitrophenyl)propionyl, N-2-methyl-2-(o-nitrophenoxy)propionyl, N-2-methyl-2-(o-phenylazophenoxy)propionyl, N-4-chlorobutyryl, N-3-methyl-3-nitrobutyryl, N-o-nitrocinnamoyl, N-acetylmethionine derivative, N-o-nitrobenzoyl, N-o-(benzoyloxymethyl)benzoyl, and 4,5-diphenyl-3-oxazolin-2-one.

Cyclic Imide Derivatives

N-phthalimide, N-dithiasuccinoyl, N-2,3-diphenylmaleoyl, N-2,5-dimethylpyrrolyl, N-1,1,4,4-tetramethyldisilylazacyclopentane adduct, 5-substituted 1,3-dimethyl-1,3,5-triazacyclohexan-2-one, 5-substituted 1,3-dibenzyl-1,3,5-triazacyclohexan-2-one, and 1-substituted 3,5-dinitro-4-pyridonyl. Examples of special NH protective groups include

N-Alkyl and N-Aryl Amines

N-methyl, N-allyl, N-[2-(trimethylsilyl)ethoxy]methyl, N-3-acetoxypropyl, N-(1-isopropyl-4-nitro-2-oxo-3-pyrrolin-3-yl), quaternary ammonium salts, N-benzyl, N-di(4-methoxyphenyl)methyl, N-5-dibenzosuberyl, N-triphenylmethyl, N-(4-methoxyphenyl)diphenylmethyl, N-9-phenylfluorenyl, N-2,7-dichloro-9-fluorenylmethylene, N-ferrocenylmethyl, and N-2-picolylamine N′-oxide.

Imine Derivatives

N-1,1-dimethylthiomethylene, N-benzylidene, N-p-methoxybenzylidene, N-diphenylmethylene, N-[(2-pyridyl)mesityl]methylene, and N-(N′,N′-dimethylaminomethylene).

Protection for the Carbonyl Group

Acyclic Acetals and Ketals

Examples of acyclic acetals and ketals include dimethyl, bis(2,2,2-trichloroethyl), dibenzyl, bis(2-nitrobenzyl) and diacetyl.

Cyclic Acetals and Ketals

Examples of cyclic acetals and ketals include 1,3-dioxanes, 5-methylene-1,3-dioxane, 5,5-dibromo-1,3-dioxane, 5-(2-pyridyl)-1,3-dioxane, 1,3-dioxolanes, 4-bromomethyl-1,3-dioxolane, 4-(3-butenyl)-1,3-dioxolane, 4-phenyl-1,3-dioxolane, 4-(2-nitrophenyl)-1,3-dioxolane, 4,5-dimethoxymethyl-1,3-dioxolane, O,O′-phenylenedioxy and 1,5-dihydro-3H-2,4-benzodioxepin.

Acyclic Dithio Acetals and Ketals

Examples of acyclic dithio acetals and ketals include S,S′-dimethyl, S,S′-diethyl, S,S′-dipropyl, S,S′-dibutyl, S,S′-dipentyl, S,S′-diphenyl, S,S′-dibenzyl and S,S′-diacetyl.

Cyclic Dithio Acetals and Ketals

Examples of cyclic dithio acetals and ketals include 1,3-dithiane, 1,3-dithiolane and 1,5-dihydro-3H-2,4-benzodithiepin.

Acyclic Monothio Acetals and Ketals

Examples of acyclic monothio acetals and ketals include O-trimethylsilyl-S-alkyl, O-methyl-S-alkyl or -S-phenyl and O-methyl-S-2-(methylthio)ethyl.

Cyclic Monothio Acetals and Ketals

Examples of cyclic monothio acetals and ketals include 1,3-oxathiolanes.

Miscellaneous Derivatives

O-Substituted Cyanohydrins

Examples of O-substituted cyanohydrins include O-acetyl, O-trimethylsilyl, 0-1-ethoxyethyl and 0-tetrahydropyranyl.

Substituted Hydrazones

Examples of substituted hydrazones include N,N-dimethyl and 2,4-dinitrophenyl.

Oxime Derivatives

Examples of oxime derivatives include O-methyl, O-benzyl and O-phenylthiomethyl.

Imines

Substituted Methylene Derivatives, Cyclic Derivatives

Examples of substituted methylene and cyclic derivatives include oxazolidines, 1-methyl-2-(1′-hydroxyalkyl)imidazoles, N,N′-dimethylimidazolidines, 2,3-dihydro-1,3-benzothiazoles, diethylamine adducts, and methylaluminum bis(2,6-di-t-butyl-4-methylphenoxide)(MAD)complex.

Monoprotection of Dicarbonyl Compounds

Selective Protection of α-and β-Diketones

Examples of selective protection of α- and β-diketones include enamines, enol acetates, enol ethers, methyl, ethyl, i-butyl, piperidinyl, morpholinyl, 4-methyl-1,3-dioxolanyl, pyrrolidinyl, benzyl, S-butyl, and trimethylsilyl.

Cyclic Ketals. Monothio and Dithio Ketals

Examples of cyclic ketals, monothio and dithio ketals include bismethylenedioxy derivatives and tetramethylbismethylenedioxy derivatives.

Protection for the Carboxyl Group

Esters

Substituted Methyl Esters

Examples of substituted methyl esters include 9-fluorenylmethyl, methoxymethyl, methylthiomethyl, tetrahydropyranyl, tetrahydrofuranyl, methoxyethoxymethyl, 2-(trimethylsilyl)ethoxymethyl, benzyloxymethyl, phenacyl, p-bromophenacyl, α-methylphenacyl, p-methoxyphenacyl, carboxamidomethyl, and N-phthalimidomethyl.

2-Substituted Ethyl Esters

Examples of 2-substituted ethyl esters include 2,2,2-trichloroethyl, 2-haloethyl, ω-chloroalkyl, 2-(trimethylsilyl)ethyl, 2-methylthioethyl, 1,3-dithianyl-2-methyl, 2-(p-nitrophenylsulfenyl)ethyl, 2-(p-toluenesulfonyl)ethyl, 2-(2′-pyridyl)ethyl, 2-(diphenylphosphino)ethyl, 1-methyl-1-phenylethyl, t-butyl, cyclopentyl, cyclohexyl, allyl, 3-buten-1-yl, 4-(trimethylsilyl)-2-buten-1-yl, cinnamyl, α-methylcinnamyl, phenyl, p-(methylmercapto)phenyl and benzyl.

Substituted Benzyl Esters

Examples of substituted benzyl esters include triphenylmethyl, diphenylmethyl, bis(o-nitrophenyl)methyl, 9-anthrylmethyl, 2-(9,10-dioxo)anthrylmethyl, 5-dibenzosuberyl, 1-pyrenylmethyl, 2-(trifluoromethyl)-6-chromylmethyl, 2,4,6-trimethylbenzyl, p-bromobenzyl, o-nitrobenzyl, p-nitrobenzyl, p-methoxybenzyl, 2,6-dimethoxybenzyl, 4-(methylsulfinyl)benzyl, 4-sulfobenzyl, piperonyl, 4-picolyl and p-P-benzyl.

Silyl Esters

Examples of silyl esters include trimethylsilyl, triethylsilyl, t-butyldimethylsilyl, i-propyidimethylsilyl, phenyidimethylsilyl and di-t-butylmethylsilyl.

Activated Esters

Examples of activated esters include thiols.

Miscellaneous Derivatives

Examples of miscellaneous derivatives include oxazoles, 2-alkyl-1,3-oxazolines, 4-alkyl-5-oxo-1,3-oxazolidines, 5-alkyl-4-oxo-1,3-dioxolanes, ortho esters, phenyl group and pentaaminocobalt(III) complex.

Stannyl Esters

Examples of stannyl esters include triethylstannyl and tri-n-butylstannyl.

Amides

Examples of amides include N,N-dimethyl, pyrrolidinyl, piperidinyl, 5,6-dihydrophenanthridinyl, o-nitroanilides, N-7-nitroindolyl, N-8-Nitro-1,2,3,4-tetrahydroquinolyl, and p-P-benzenesulfonamides.

Hydrazides

Examples of hydrazides include N-phenyl and N,N′-diisopropyl.

### E. Chemical Examples

### Example 1

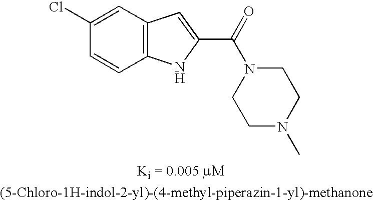

A mixture of 5-chloroindole-2-carboxylic acid (0.234 g), HATU (0.569 g), HOAT (0.203 g) and N, N-diisopropylethylamine (0.191 mL) in DMF (0.6 mL) was treated with N-methylpiperazine (0.1 mL) stirred at ambient temperature for 48 h then concentrated under reduced pressure. The residue was dissolved in ethyl acetate, washed with 1 M hydrochloric acid, saturated sodium hydrogen carbonate solution and then brine, dried over sodium sulfate, filtered, and concentrated under reduced pressure. The residue was purified via silica gel chromatography (3-10% 2 M ammonia in methanol/dichloromethane) to give the title compound (0.18 g). ¹H NMR (400 MHz, CDCl₃): δ9.60 (br s,1 H), 7.65 (d, J=1.5 Hz, 1H), 7.40 (d, J=8.6 Hz, 1H), 7.29 (d, J=2.0 Hz, 1H), 7.26 (d, 1.8 Hz, 1H), 6.76 (d, J=1.5 Hz, 1H), 4.0 (br m, 4H), 2.56 (t, J=5.1 Hz, 4H), 2.41 (s, 3H). Analysis: Calc'd for C₁₄H₁₆ClN₃O; C, 60.54; H, 5.81; N, 15.13; Found: C, 59.99; H, 5.94; N, 18.87. The title compounds of the following examples (2-14) were prepared according to the general procedure of Scheme 1, as indicated for Example 1.

### Example 2

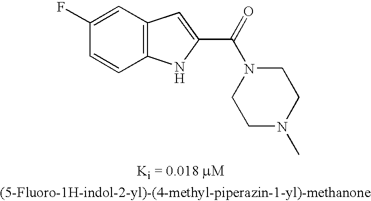

¹H NMR (400 MHz, CDCl₃): δ9.70 (br s, 1H), 7.33 (m, 2H), 7.09-6.98 (m, 1H), 6.75 (m, 1H), 3.97 (br m, 4H), 2.53 (dm, J=4.7 Hz, 4H), 2.38 (s, 3H).

### Example 3

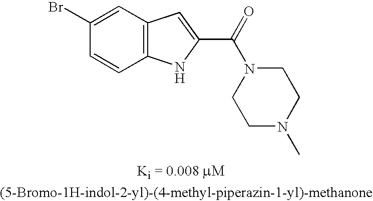

¹H NMR (400 MHz, CDCl₃): δ9.65 (br s, 1H), 7.78 (d, J=1.0 Hz, 1H), 7.40-7.26 (m, 2H), 6.73 (d, J=2.3 Hz, 1H), 3.97 (br m, 4H), 2.53 (t, J=5.1 Hz, 4H), 2.37 (s, 3H).

### Example 4

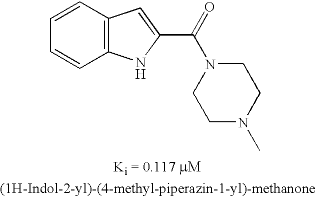

¹H NMR (400 MHz, CDCl_{3/}CD₃OD): δ7.63-7.56 (m,1H), 7.40 (dt, J=1.0, 8.3 Hz, 1H), 7.26-7.20 (m, 1H), 7.11-7.05 (m, 1H), 6.99 (d, J=0.8 Hz), 6.72 (d, J=0.8 Hz), 3.88 (br m, 4H), 2.48 (t, J=5.1 Hz, 4H), 2.31 (s, 3H).

### Example 5

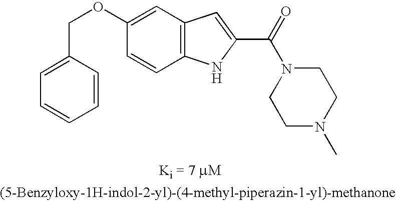

### Example 6

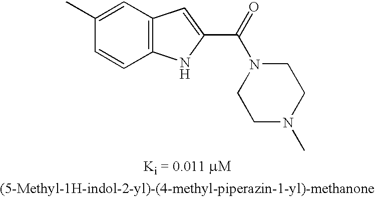

¹H NMR (400 MHz, CDCl₃): δ8.91 (br s, 1H), 7.34 (dm, J=0.7 Hz, 1H), 7.24 (d, J=8.3 Hz, 1H), 7.04 (dd, J=8.3, 1.3 Hz, 1H), 6.62 (dd, J=2.0, 0.8 Hz, 1H), 3.88 (br m, 4H), 2.44 (t, J=4.0 Hz, 4H), 2.37 (s, 3H), 2.29 (s, 3H).

### Example 7

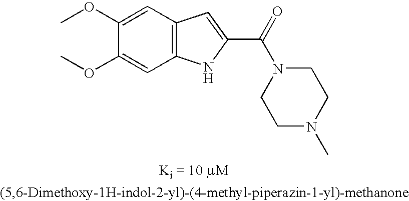

### Example 8

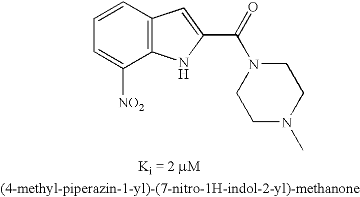

¹H NMR (400 MHz, CDCl₃): δ10.46 (brs, 1H), 8.29 (d, 1H), 8.06 (d, 1H), 7.34 (m, 1H), (t, 1H), 3.94 (br m, 4H), 2.54 (t, 4H), 2.40 (s, 3H).

### Example 9

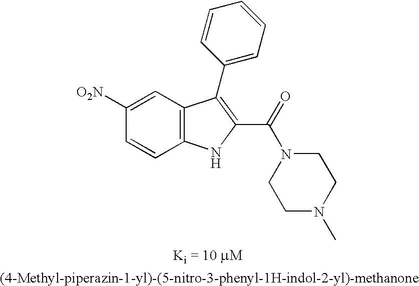

### Example 10

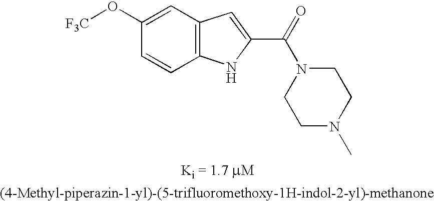

### Example 11

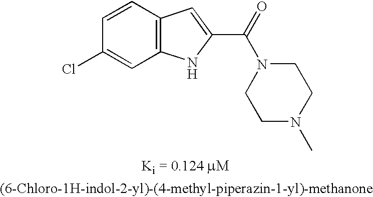

¹H NMR (400 MHz, CDCl₃): δ10.14 (br s, 1H), δ7.55 (d, J=8.3 Hz, 1H), 7.44 (t, J=1.0 Hz, 1H), 7.10 (dd, J=8.3, 1.8 Hz, 1H), 6.76 (dd, J=2.3, 1.0 Hz, 1H), 4.00 (br m, 4H), 2.54 (t, J=5.1 Hz, 4H), 2.38 (s, 3H). MS: exact mass calculated for C₁₄H₁₆ClN₃O, 277.10; m/z found, 278.1 [M+H]⁺.

### Example 12

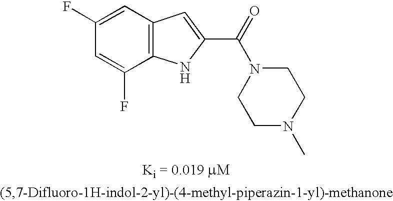

¹H NMR (400 MHz, CDCl₃): δ9.94 (br s,1H), 7.10 (dd, J=8.8, 2.0 Hz, 1H), 6.87-6.78 (m, 1H), 6.77 (t, J=2.8 Hz, 1H), 3.97 (br m, 4H), 2.53 (t, J=5.1 Hz, 4H), 2.37 (s, 3H). MS: exact mass calculated for C₁₄H₁₅F₂N₃O, 279.12; m/z found, 280 [M+H]⁺.

### Example 13

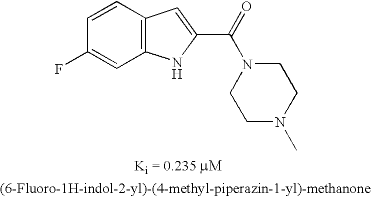

¹H NMR (400 MHz, CDCl₃): δ9.45 (br s, 1H), 7.49 (dd, J=8.8, 5.6 Hz, 1H), 7.02 (dd, J=, 9.4, 2.3 Hz, 1H), 6.87-6.81 (m,1H), 6.69 (dd, J=2.0, 1.0 Hz, 1H), 3.89 (br m, 4H), 2.44 (t, J=5.1 Hz, 4H), 2.88 (s, 3H).

### Example 14

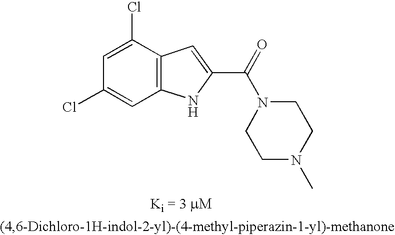

### Example 15

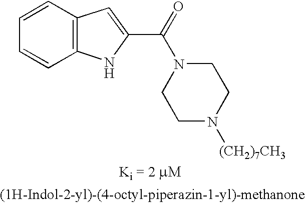

Indole-2-carboxylic acid (0.193 g) in THF (25 mL) was treated with carbonyldiimidazole (0.178 g) and stirred at ambient temperature for 2 h whereupon 1-octyl-piperazine (0.142 g) was added. The mixture was stirred at ambient temperature for 18 h, and the solvent removed under reduced pressure. The residue was dissolved in ethyl acetate and washed with saturated sodium bicarbonate solution, and the organic portion was separated, dried over sodium sulfate and filtered. Solvent was evaporated to afford the title compound (0.28 g). ¹H NMR (400 MHz, CD₃OD): δ7.50 (d, J=8.0 Hz, 1H), 7.32 (d, J=8.3 Hz, 1H), 7.13-7.09 (m, 1H), 6.98-6.94 (m, 1H), 6.71 (s, 1H), 3.79 (s, 4H), 2.46 (t, J=4.7 Hz, 4H), 2.32 (t, J=7.7 Hz, 2H), 1.46 (br s, 2H), 1.36-1.03 (m, 12H), 0.82-0.79 (m, 3H).

The title compounds of the following examples (16-38) were prepared according to the general procedure of Scheme 1, as indicated for Example 15.

### Example 16

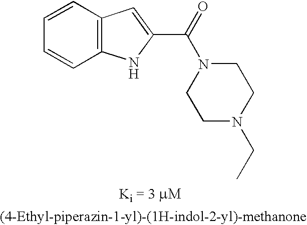

### Example 17

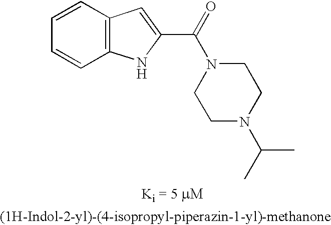

### Example 18

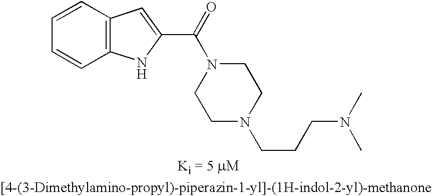

### Example 19

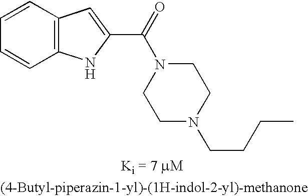

### Example 20

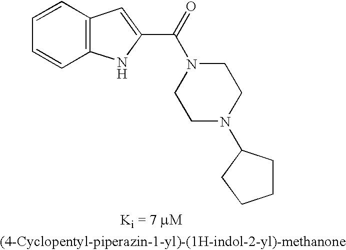

### Example 21

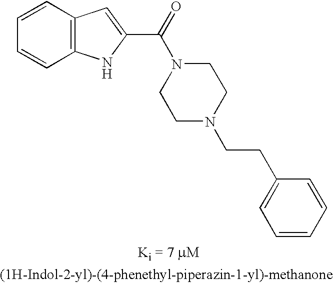

### Example 22

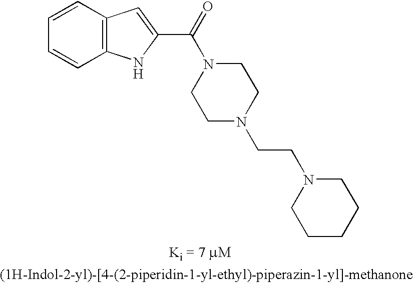

### Example 23

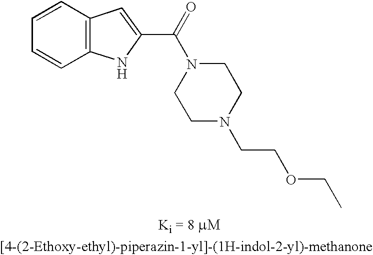

### Example 24

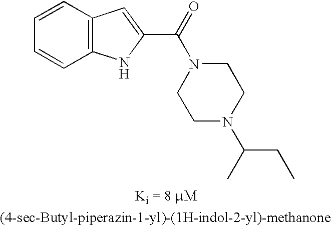

### Example 25

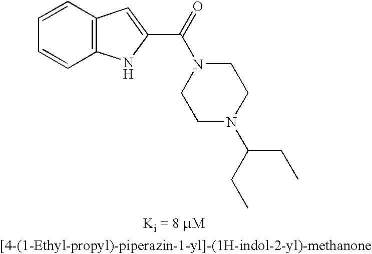

### Example 26

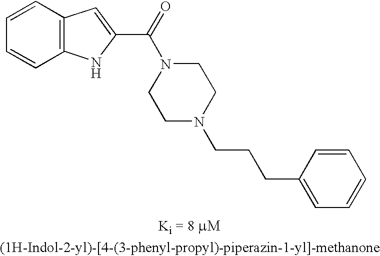

### Example 27

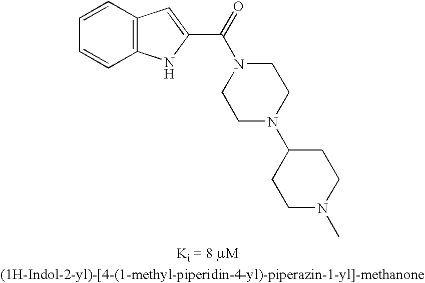

### Example 28

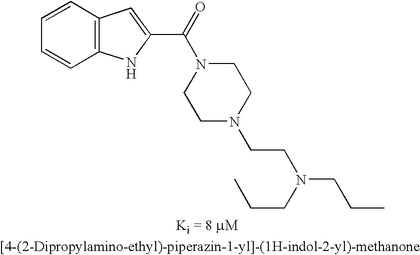

### Example 29

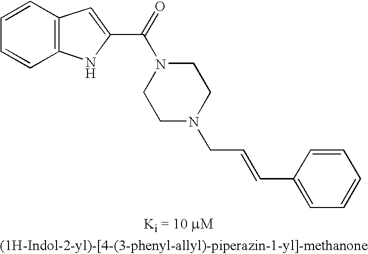

### Example 30

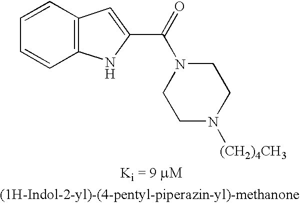

### Example 31

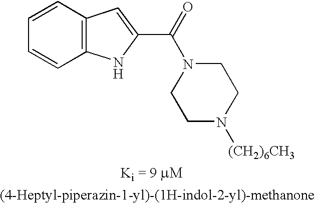

### Example 32

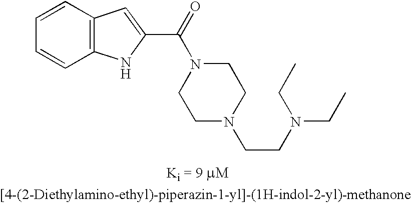

### Example 33

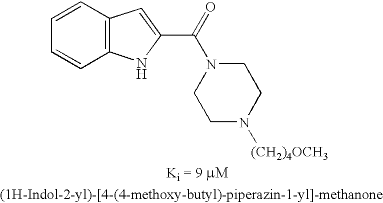

### Example 34

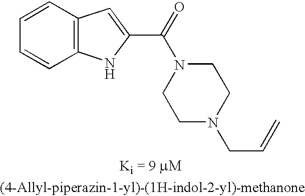

### Example 35

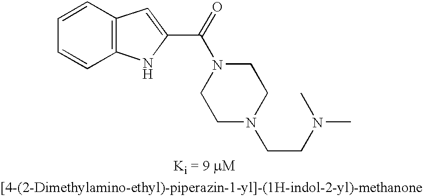

### Example 36

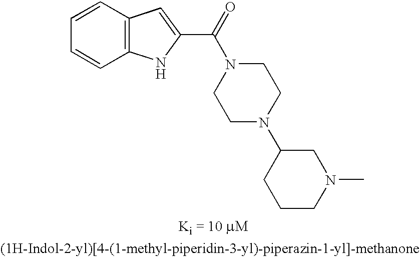

### Example 37

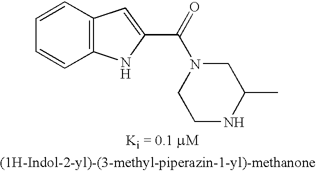

¹H NMR (400 MHz, CDCl₃): δ9.16 (s, 1H), 7.65 (d, J=7.9 Hz, 1H), 7.42 (d, J=8.3 Hz, 1H), 7.30-7.25 (m, 1H), 7.14 (t, J=7.2 Hz, 1H), 6.77 (s, 1H), 4.59 (m, 2H), 3.10 (m, 1H), 2.94-2.86 (m, 2H), 1.65 (s, 3H), 1.14 (d, J=5.6 Hz, 3H).

### Example 38

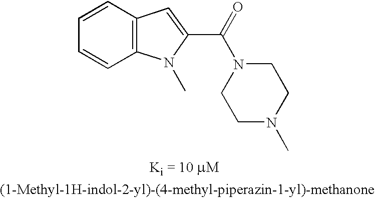

¹H NMR (400 MHz, CDCl₃): δ7.64 (dt, J=1.0, 7.8 Hz,1H), 7.38 (dd, J=8.3, 0.8 Hz, 1H), 7.35-7.32 (m, 1H), 7.19-7.14 (m, 1H), 6.62 (d, J=0.8 Hz, 1H), 3.86 (s, 3H), 3.83 (br m, 4H), 2.49 (br m, 4H), 2.37 (s, 3H).

### Example 39

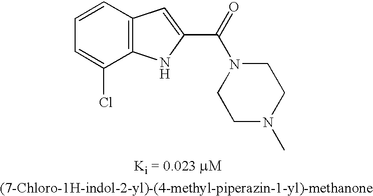

2-Chlorophenylhydrazine hydrochloride (0.5 g) in ethanol (25 mL) was treated with ethylpyruvate (0.324 g) and concentrated sulfuric acid (3 drops). The mixture was stirred at ambient temperature for five min and treated with polyphosphoric acid (0.5 g). The mixture was heated at reflux temperature for 24 h whereupon additional polyphosphoric acid (0.5 g) was added and heating continued for a further 48 h. The reaction mixture was cooled to ambient temperature and concentrated under reduced pressure. The residue was partitioned between ethyl acetate and water and the pH of the aqueous layer adjusted to neutrality by addition of saturated sodium hydrogen carbonate solution. The organic fraction was separated, washed with brine, dried over magnesium sulfate, filtered, and concentrated. The residue was purified via silica gel chromatography (5-10% ethyl acetate/hexane) to give 7-Chloro-1H-indole-2-carboxylic acid ethyl ester (0.227 g). This material (0.102 g) was used without further purification. The ester was treated with 1 M lithium hydroxide in ethanol (5 mL) followed by water (3 mL) and stirred at ambient temperature for 18 h. The solution was acidified with 10% hydrochloric acid, diluted with water and extracted with ethyl acetate. The organic extracts were washed with brine, dried over magnesium sulfate, filtered, and concentrated to afford give 7-Chloro-1H-indole-2-carboxylic acid (0.089 g). This material (0.089 g), was treated with HATU (0.259 g), HOAT (0.093 g), N,N-diisopropylethylamine (0.158 mL) and N-methylpiperazine (0.05 mL) in DMF (0.6 mL) and stirred at ambient temperature for 18 h. The reaction mixture was concentrated under reduced pressure. The residue was dissolved in ethyl acetate, washed with 1 M hydrochloric acid, saturated sodium hydrogencarbonate solution and then brine, dried over magnesium sulfate, filtered, and concentrated under reduced pressure. The residue was purified via silica gel chromatography (2-10% 2 M ammonia in methanol/dichloromethane) to give the title compound (0.56 g). ¹H NMR (400 MHz, CDCl₃): δ9.17 (br s, 1H), 7.47 (d, J=8.1 Hz, 1H), 7.21 (dd, J=7.6, 0.8 Hz, 1H), 7.01 (t, J=7.8 Hz, 1H), 6.73 (d, J=2.3 Hz, 1H), 3.88 (br m, 4H), 2.45 (t, J=5.1 Hz, 4H), 2.29 (s, 3H).

The title compounds of the following examples (40-43) were prepared according to the general procedure of Scheme 2, as indicated for example 39.

### Example 40

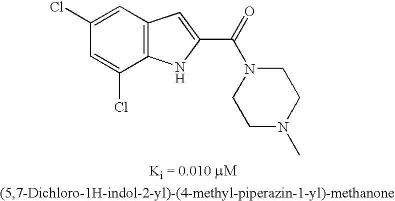

¹H NMR (400 MHz, CDCl₃): δ9.08 (br s, 1H), 7.36 (dd, J=1.8, 0.8 Hz, 1H), 7.12 (d, J=1.8 Hz, 1H), 6.56 (d, J=2.3 Hz, 1H), 3.77 (br m, 4H), 2.34 (t, J=5.1 Hz, 4H), 2.20 (s, 3H).

### Example 41

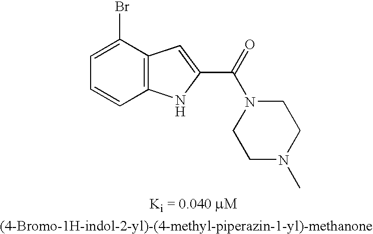

### Example 42

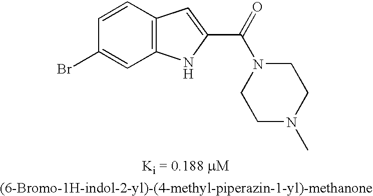

¹H NMR (400 MHz, CDCl₃): δ9.70 (br s, 1H), 7.69 (t, J=0.8 Hz, 1H), 7.51 (d, J=8.6 Hz, 1H), 7.24 (dd, J=8.6, 1.8 Hz, 1H), 6.76 (dd, J 2.0, 1.0 Hz, 1H), 3.98 (br m, 4H), 2.54 (t, J=5.1 Hz, 4H), 2.37 (s, 3H).

### Example 43

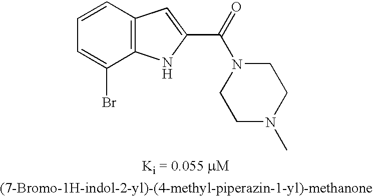

¹H NMR (400 MHz, CDCl₃): δ9.06 (br s, 1H), 7.51 (dt, J=0.8, 8.1 Hz, 1H), 7.36 (dd, J=7.7, 0.8 Hz, 1H), 6.96 (t, J=7.8 Hz, 1H), 6.76 (d, J=2.3 Hz, 1H), 3.87 (br m, 4H), 2.43 (t, J=5.1 Hz, 4H), 2.28 (s, 3H). MS: exact mass calculated for C₁₄H₁₆BrN₃O, 321.05; m/z found, 322.1 [M+H]⁺.

The title compound of the following example (44) was prepared according to the general procedure of Scheme 3.

### Example 44

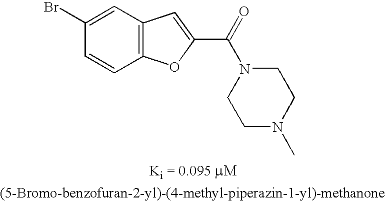

5-Bromo-benzofuran-2-carboxylic acid (0.346 g) in THF (7 mL) was treated with carbonyldiimidazole (0.214 g) and stirred at ambient temperature for 2 h whereupon methyl-piperazine (0.129 g) was added. The mixture was stirred at ambient temperature for 18 h and then concentrated under reduced pressure. The residue was dissolved in dichloromethane and washed with saturated sodium bicarbonate solution, whereupon the organic portion was separated out, dried over sodium sulfate and filtered. The solvent was evaporated, and the residue was purified via silica gel chromatography (5% 2M ammonina in methanol/dichloromethane) to afford the title compound (0.222 g). ¹H NMR (400 MHz, CDCl₃): δ7.75 (d, J=1.9 Hz, 1H), 7.45 (dd, J=8.8, 1.9 Hz,1H), 7.37 (d, J=8.8 Hz, 1H), 3.83 (br s, 4H), 2.48 (t, J=4.8 Hz, 4H), 2.33 (s, 3H). ¹³C NMR (400 MHz, CDCl₃) δ159.4, 153.4, 150.3, 129.6, 129.0, 124.9, 116.8, 113.5, 111.3, 55.3, 54.9, 46.8, 46.1, 42.9.

### Example 45

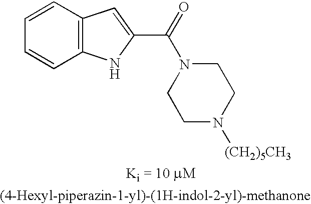

Indole-2-carboxylic acid (5.2 g) in THF (200 mL) was treated with carbonyldiimidazole (4.8 g) and stirred at ambient temperature for 10 min whereupon 4-methyl-piperazine-1-carboxylic acid tert-butyl ester (5.0 g) was added. The mixture was stirred at ambient temperature for 72 h and the solvent removed under reduced pressure. The residue was dissolved in ethyl acetate and washed with saturated sodium bicarbonate solution. The organic portion was separated, dried over sodium sulfate and filtered, and solvent was evaporated to afford a solid. Recrystallization from hot ethanol afforded 4-(1H-Indole-2-carbonyl)-piperazine-1-carboxylic acid tert-butyl ester (4.2 g).

4-(1H-Indole-2-carbonyl)-piperazine-1-carboxylic acid tert-butyl ester (0.165 g) in dichloromethane (10 mL) was treated with trifluoroacetic acid (2 mL) and stirred at ambient temperature for 1 h. The solvent was removed under reduced pressure to afford (1H-Indol-2-yl)-piperazin-1-yl-methanone trifluoroacetate salt. (¹H NMR (400 MHz, CDCl₃): δ7.63 (d, J=8.07 Hz, 1H), 7.44 (dd, J=8.3, 0.8 Hz, 1H), 7.24 (m, 1H), 7.08 (m, 1H), 6.91 (s, 1H), 4.12 (t, J=5.0 Hz, 4H), 3.35 (t, J=5.3 Hz, 4H)). This intermediate was dissolved in acetone (5 mL), treated with potassium carbonate (0.22 g), iodohexane (0.106 g) and heated at 50° C. for 10 h. Evaporation of the solvent under reduced pressure afforded crude product which was purified via preparative thin layer chromatography eluting with 10% methanol/dichloromethane to give the title compound (0.06 g). ¹H NMR (400 MHz, CD₃OD): δ7.60 (d, J=8.0 Hz, 1H), 7.42 (d, J=8.3 Hz, 1H), 7.21(ddd, J=8.1, 7.1, 1.1 Hz, 1H), 7.16-7.04 (m, 1H), 6.81 (s, 1H), 3.89 (br s, 4H), 2.56 (t, J=5.0 Hz, 4H), 2.43-2.40 (m, 2H), 1.58-1.52 (m, 2H), 1.34 (br s, 6H), 0.94-0.90 (m, 3H).

The title compounds of the following examples (46-47) were prepared according to the general procedure of Scheme 5, as indicated for Example 74.

### Example 46

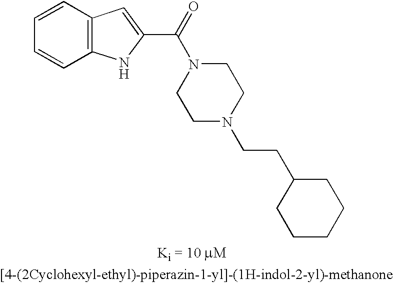

### Example 47

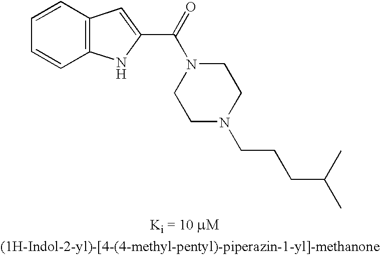

### Example 48

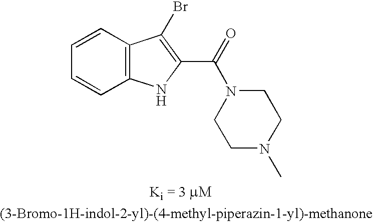

(1H-Indol-2-yl)-(4-methyl-piperazin-1-yl)-methanone (Example 4, 0.222 g) in acetic acid (1 mL) at ambient temperature was treated with bromine (0.05 mL) and stirred for 7 h. The reaction mixture was poured into water and adjusted to basic pH by addition of 1 M sodium hydroxide. The mixture was extracted with dichloromethane. The organic extracts were combined, dried over sodium sulfate, filtered, and concentrated to give crude product. Purification via silica gel chromatography, eluting with 1-8% methanol/dichloromethane, afforded the title compound (0.154 g).

### Example 49

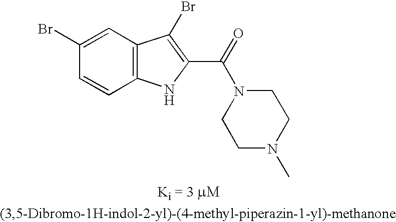

(1H-Indol-2-yl)-(4-methyl-piperazin-1-yl)-methanone (Example 4, 0.222 g) in acetic acid (1 mL) at ambient temperature was treated with bromine (0.10 mL) and stirred for 7 h. The reaction mixture was poured into water and adjusted to basic pH by addition of 1 M sodium hydroxide. The mixture was extracted with dichloromethane. The organic extracts were combined, dried over sodium sulfate, filtered, and concentrated to give crude product. Purification via silica gel chromatography, eluting with 1-8% methanol/dichloromethane afforded the title compound (0.123 g).

### Example 50

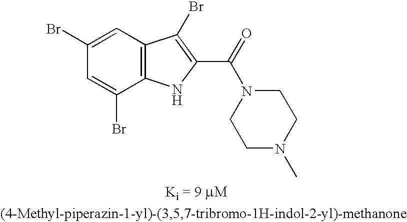

(1H-Indol-2-yl)-(4-methyl-piperazin-1-yl)-methanone (Example 4, 0.222 g) in acetic acid (1 mL) at ambient temperature was treated with bromine (0.15 mL) and stirred for 7 h. The reaction mixture was poured into water and adjusted to basic pH by addition of 1 M sodium hydroxide. The mixture was extracted with dichloromethane. The organic extracts were combined, dried over sodium sulfate, filtered, and concentrated to give crude product. Purification via silica gel chromatography, eluting with 1-8% methanol/dichloromethane afforded the title compound (0.038 g).

### Example 51

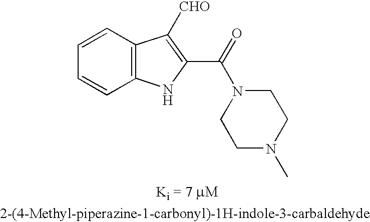

(1H-Indol-2-yl)-(4-methyl-piperazin-1-yl)-methanone (Example 4, 0.206 g) in DMF (1.5 mL) at 0° C. was treated with phosphorus oxychloride (0.1 mL) over 10 min. The reaction mixture was warmed to ambient temperature and stirred for 16 h. The reaction mixture was poured into water and adjusted to neutral pH by addition of 1 M sodium hydroxide. The mixture was extracted with dichloromethane. The organic extracts were combined, dried over sodium sulfate, filtered, and concentrated to give crude product. Purification via silica gel chromatography, eluting with 1-8% methanol/dichloromethane afforded the title compound (0.108 g).

### Example 52

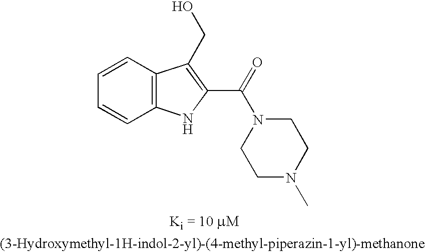

2-(4-Methyl-piperazine-1-carbonyl)-1H-indole-3-carbaldehyde (Example 51, 0.094 g) in ethyl acetate (1.5 mL) was treated with sodium borohydride (0.024 g) and stirred at ambient temperature for 3 h. The solvent was removed under reduced pressure, and the residue treated with saturated sodium hydrogencarbonate solution and extracted with dichloromethane. The organic extracts were dried over sodium sulfate, filtered, and concentrated. The residue was purified via silica gel chromatography, eluting with 1-8% methanol/dichloromethane, to afford the title compound (0.042 g).

### Example 53

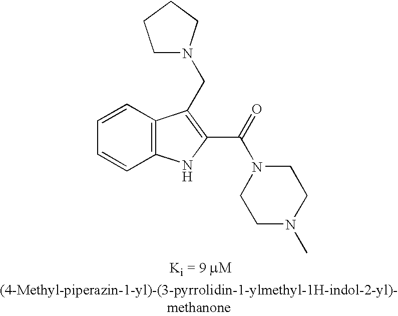

(1H-Indol-2-yl)-(4-methyl-piperazin-1-yl)-methanone (Example 4, 0.231 g) in acetic acid (1.5 mL) at ambient temperature was treated with paraformaldehyde (0.4 g) and pyrrolidine (0.16 mL). The reaction mixture was heated at 60° for 6 h then poured into water and the solution adjusted to basic pH by addition of 1 M sodium hydroxide. The mixture was extracted with dichloromethane. The organic extracts were combined, dried over sodium sulfate, filtered, and concentrated to give crude product. Purification via silica gel chromatography, eluting with 1-8% methanol/dichloromethane afforded the title compound (0.1 g).

### Example 54

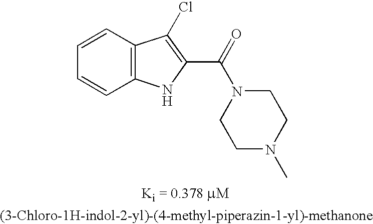

(1H-Indol-2-yl)-(4-methyl-piperazin-1-yl)-methanone (Example 4, 0.5 g) in dichloromethane (3 mL) at ambient temperature was treated with N-chlorosuccinimide (0.301 g) and stirred for 6 h. The reaction mixture was diluted with ether, washed with water, saturated sodium hydrogencarbonate solution and then brine, dried over sodium sulfate, filtered, and concentrated to give crude product. Purification via silica gel chromatography, eluting with 1-8% methanol/dichloromethane, afforded the title compound (0.36 g). ¹H NMR (400 MHz, CDCl₃): δ2.36 (3H), 2.52 (4H), 3.79 (4H), 7.21 (1H), 7.31 (1H), 7.38 (1H), 7.64 (1H), 9.05 (1H).

### Example 55

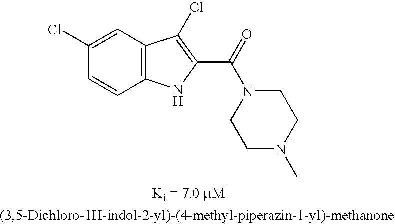

(5-Chloro-1H-indol-2-yl)-(4-methyl-piperazin-1-yl)-methanone (Example 1, 0.23 g) in dichloromethane (3 mL) at ambient temperature was treated with N-chlorosuccinimide (0.123 g) and stirred for 18 h. The reaction mixture was diluted with ether, washed with water, saturated sodium hydrogencarbonate solution and then brine, dried over sodium sulfate, filtered, and concentrated to give crude product. Purification via silica gel chromatography, eluting with 1-8% methanol/dichloromethane afforded the title compound (0.13 g). ¹H NMR (400 MHz, CDCl₃): δ2.36 (3H), 2.53 (4H), 3.79 (4H), 7.22 (1H), 7.29 (1H), 7.58 (1H), 10.39 (1H).

### Example 56

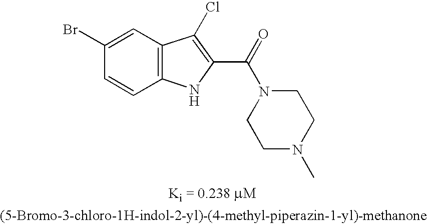

(5-Bromo-1H-indol-2-yl)-(4-methyl-piperazin-1-yl)-methanone (Example 3, 0.27 g) in dichloromethane (3 mL) at ambient temperature was treated with N-chlorosuccinimide (0.103 g) and stirred for 18 h. The reaction mixture was diluted with ether, washed with water, saturated sodium hydrogencarbonate solution and then brine, dried over sodium sulfate, filtered, and concentrated to give crude product. Purification via silica gel chromatography, eluting with 1-8% methanol/dichloromethane afforded the title compound (0.16 g). ¹H NMR (400 MHz, CDCl₃): δ2.35 (3H), 2.52 (4H), 3.78 (4H), 7.23 (1H), 7.35 (1H), 7.74 (1H), 9.84 (1H).

### Example 57

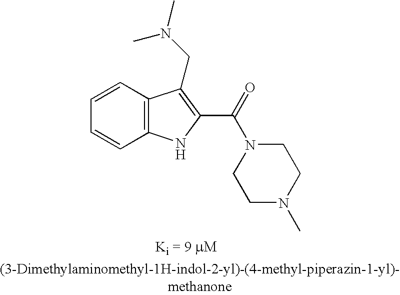

The title compound was prepared from (1H-Indol-2-yl)-(4-methyl-piperazin-1-yl)-methanone (Example 4) according to the general procedure of Example 53 (See: J. Am. Chem. Soc., 71:3541, 1949). ¹H NMR (400 MHz, CDCl₃: δ9.39 (br, 1H), 7.78 (m, 1H), 7.34 (m, 1H), 7.21 (m, 1H), 7.11 (m, 1H), 5.28 (s, 2H), 3.69 (br, 4H), 2.40 (br, 4H), 2.29 (s, 3H), 2.24 (s, 6H)). MS (electrospray): exact mass calculated for C₁₇H₂₄N₄O, 300.20; m/z found, 301.1 [M+H]⁺.

### Example 58

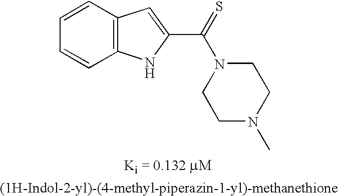

(1H-Indol-2-yl)-(4-methyl-piperazin-1-yl)-methanone (Example 4, 0.123 g) in THF (1 mL) was treated with Lawesson's reagent (0.243 g) and stirred at ambient temperature overnight. The reaction mixture was concentrated under reduced pressure, and the residue was purified via preparative thin layer chromatography to afford the title compound (0.02 g). ¹H NMR (400 MHz, CDCl₃): δ9.21 (br s, 1H), 7.62 (d, J=8.0 Hz, 1H), 7.40 (d, J=8.3 Hz, 1H), 7.29 (d, J=7.3 Hz, 1H), 7.12 (m, 1H), 6.60 (s, 1H), 4.39 (br s, 4H), 3.85 (br s, 4H), 2.63 (s, 3H).

The title compounds of the following examples (59 and 60) were prepared according to the general procedure of Scheme 1.

### Example 59

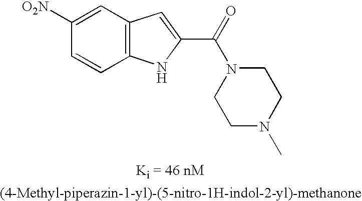

A mixture of 5-nitroindole-2-carboxylic acid (4.38 g) and 1-(3-dimethylaminopropyl)-3-ethylcarbodiimide hydrochloride (EDC, 4.89 g) in dichloromethane (150 mL) was treated with N-methylpiperazine (2.83 mL) and stirred at ambient temperature for 16 h. The reaction mixture was poured into dichloromethane (200 mL), washed with water, saturated sodium hydrogencarbonate solution and then brine, dried over sodium sulfate, filtered, and concentrated under reduced pressure. The residue was purified via silica gel chromatography (0-10% 2M ammonia in methanol/dichloromethane) to give the title compound (1.8 g). ¹H NMR (400 MHz, CDCl₃): δ10.97 (br s, 1H), 8.58 (d, J=2.15 Hz, 1H), 8.11 (dd, J=2.15, 7.04 Hz, 1H), 7.44 (d, J=9.00 Hz, 1H), 6.89 (s, 1H), 3.95 (br m, 4H), 2.52 (t, J=4.89 Hz, 4H), 2.34 (s, 3H). MS (electrospray): exact mass calculated for C₁₄H₁₆N₄O₃, 288.12; m/z found, 289.1 [M+H]⁺.

### Example 60

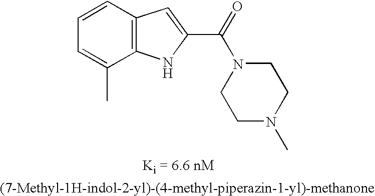

A mixture of 7-methylindole-2-carboxylic acid (1.79 g, 10 mmol), 1-(3-dimethylaminopropyl)-3-ethylcarbodiimide hydrochloride (EDC, 2.88 g , 15 mmol) in CH₂Cl₂ (100 mL) was treated with N-methylpiperazine (2.22 mL, 20 mmol). The reaction mixture was stirred at ambient temperature for 16 h and then concentrated under reduced pressure. The residue was dissolved in CH₂Cl₂ (100 mL), washed with water (25 mL ×2) and then brine (25 mL), dried over sodium sulfate, filtered, and concentrated under reduced pressure. This product was purified via silica gel chromatography (5-10% methanol/dichloromethane) to give the title compound as a white solid (2.5 g, 97.3%). ¹H NMR (400 MHz, CDCl₃): δ11.07 (br s, 1H), 7.43 (d, J=7.04 Hz, 1H), 7.00-6.92 (m, 2H), 6.71 (d, J=1.96 Hz, 1H), 3.86 (br s, 4H), 2.37 (s, 3H), 2.35-2.28 (m, 4H), 2.19 (s, 3H). MS (electrospray): exact mass calculated for C₁₅H₁₉N₃O, 257.15; m/z found, 258.2 [M+H]⁺.

### Example 61

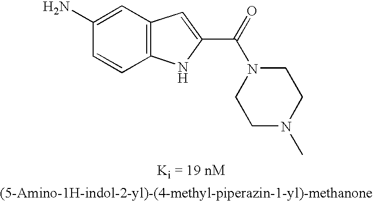

The product of Example 59, (4-Methyl-piperazin-1-yl)-(5-nitro-1H-indol-2-yl)-methanone (1.8 g) was dissolved in CH₃OH (50 mL). At room temperature, ammonium formate (3.94 g) was added, followed by 10% palladium on carbon (0.66 g). The reaction mixture was heated to reflux for forty min, cooled and filtered through celite pad. The filtrate was concentrated and the residue was purified via silica gel chromatography (3-10% 2 M ammonia in methanol/dichloromethane) to give the title compound (1.60 g). ¹H NMR (400 MHz, CDCl₃): δ10.46 (brs, 1H), 7.12 (d, J=8.80 Hz, 1H), 6.81 (d, J=2.15 Hz, 1H), 6.64 (dd, J=2.15, 6.46 Hz, 1H), 6.54 (d, J=1.37 Hz, 1H), 3.88 (br m, 4H), 3.70 (br s, 2H), 2.40 (t, J=4.70 Hz, 4H), 2.25 (s, 3H). MS (electrospray): exact mass calculated for C₁₄H₁₈N₄O, 258.15; m/z found, 259.1 [M+H]⁺.

### Example 62

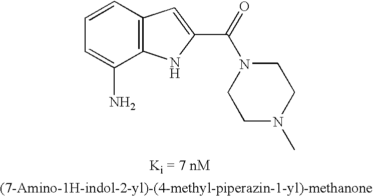

The product of Example 8, (4-Methyl-piperazin-1-yl)-(7-nitro-1H-indol-2-yl)-methanone (6.4 g, 22.2 mmol), was dissolved in CH₃OH (110 mL). At room temperature, ammonium formate (14.0 g, 222 mmol) was added, followed by 10% palladium on carbon (2.4 g, 2.22 mmol). The reaction mixture was heated to reflux for forty min, cooled, and then filtered through a celite pad. The filtrate was concentrated, and the residue was purified via silica gel chromatography (3-10% 2 M ammonia in methanol/dichloromethane) to give the title compound (4.4 g, 76.7%) as an off-white solid. ¹H NMR (400 MHz, CDCl₃/CD₃OD): δ7.08 (d, J=7.83 Hz, 1H), 6.94 (t, J=7.83 Hz, 1H), 6.73 (s, 1H), 6.58 (d, J=7.63 Hz, 1H), 4.12 (s, 2H), 3.92 (br s, 4H), 2.51 (br s, 4H), 2.34 (s, 3H). MS (electrospray): exact mass calculated for C₁₄H₁₈N₄O, 258.15; m/z found, 259.1 [M+H]⁺.

The title compounds of the following examples (63 through 66) were prepared according to the general procedure of Scheme 1.

### Example 63

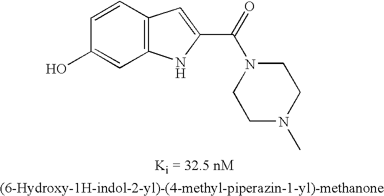

6-Methoxy-1H-indole-2-carboxylic acid ethyl ester (5.0 g) was treated with lithium hydroxide (2.33 g) in THF (90 mL) followed by water (30 mL) and stirred at ambient temperature for 16 h. The solution was acidified with 10% hydrochloric acid, diluted with water and extracted with ethyl acetate. The organic extracts were washed with brine, dried over sodium sulfate, filtered, and concentrated to afford 6-Methoxy-1H-indole-2-carboxylic acid (4.60 g). This material (4.64 g) and 1-(3-dimethylaminopropyl)-3-ethylcarbodiimide hydrochloride (5.60 g) in dichloromethane (200 mL) were treated with N-methylpiperazine (3.23 mL) and stirred at ambient temperature for 16 h. The reaction mixture was poured into dichloromethane (200 mL), washed with water, saturated sodium hydrogencarbonate solution and then brine, dried over sodium sulfate, filtered, and concentrated under reduced pressure. The residue was purified via silica gel chromatography (0-10% 2M ammonia in methanol/dichloromethane) to give (6-Methoxy-1H-indol-2-yl)-(4-methyl-piperazin-1-yl)-methanone (6.60 g). This material (0.16 g) was dissolved in dichloromethane (10 mL). At room temperature, 1 M boron tribromide (1.5 mL) was added dropwise. The reaction mixture was heated to reflux overnight, and then cooled, quenched with saturated sodium hydrogencarbonate solution, and extracted with dichloromethane. The organic extracts were washed with brine, dried over sodium sulfate, filtered, and concentrated. The residue was purified via silica gel chromatography (0-10% 2M ammonia in methanol/dichloromethane) to give the title compound (0.12 g). ¹H NMR (400 MHz, CDCl₃/CD₃OD): δ7.22 (d, J=8.41 Hz, 1H), 6.62 (d, J=2.15 Hz, 1H), 6.5-6.47 (m, 2H), 3.69 (br s, 4H), 2.30 (t, J=5.09 Hz, 4H), 2.13 (s, 3H). MS (electrospray): exact mass calculated for C₁₄H₁₇N₃O₂, 259.13; m/z found, 260.1 [M+H]⁺.

### Example 64

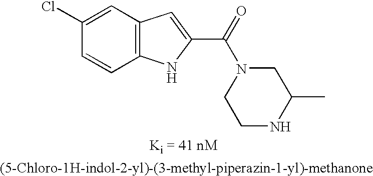

A mixture of 5-chloroindole-2-carboxylic acid (0.196 g) and 1-(3-dimethylaminopropyl)-3-ethylcarbodiimide hydrochloride (0.288 g) in dichloromethane (10 mL) was treated with 2-Methyl-piperazine (0.15 g) and stirred at ambient temperature for 16 h. The reaction mixture was poured into dichloromethane (50 mL), washed with water, saturated sodium hydrogencarbonate solution and then brine, dried over sodium sulfate, filtered and concentrated under reduced pressure. The residue was purified via silica gel chromatography (0-10% methanol/dichloromethane) to give the title compound (0.229 g). ¹H NMR (400 MHz, CDCl₃): δ10.99 (br s, 1H), 7.55 (d, J=1.76 Hz, 1H), 7.33 (d, J=8.80 Hz, 1H), 7.14 (dd, J=1.96, 6.65 Hz, 1H), 6.63 (br s, 1H), 4.55 (br s, 2H), 3.23-2.61 (m, 5H), 1.76 (br s,1H), 1.08 (d, J=5.87 Hz, 1H). MS (electrospray): exact mass calculated for C₁₄H₁₆ClN₃O, 277.10; m/z found, 278.1 [M+H]⁺.

### Example 65

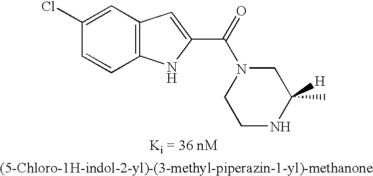

¹H NMR (400 MHz, CDCl₃): δ10.99 (br s,1H), 7.55 (d, J=1.76 Hz, 1H), 7.33 (d, J=8.80 Hz, 1H), 7.14 (dd, J=1.96, 6.65 Hz, 1H), 6.63 (br s, 1H), 4.55 (br s, 2H), 3.23-2.61 (m, 5H), 1.76 (br s, 1H), 1.08 (d, J=5.87 Hz, 1H). MS (electrospray): exact mass calculated for C₁₄H₁₆ClN₃O, 277.10; m/z found, 278.1 [M+H]⁺.

### Example 66

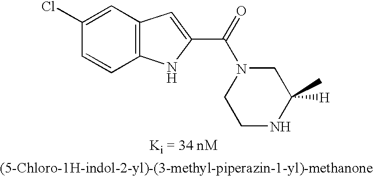

¹H NMR (400 MHz, CDCl₃): δ10.99 (br s, 1H), 7.55 (d, J=1.76 Hz, 1H), 7.33 (d, J=8.80 Hz, 1H), 7.14 (dd, J=1.96, 6.65 Hz, 1H), 6.63 (br s, 1H), 4.55 (br s, 2H), 3.23-2.61 (m, 5H), 1.76 (br s, 1H), 1.08 (d, J=5.87 Hz,1H). MS (electrospray): exact mass calculated for C₁₄H₁₆ClN₃O, 277.10; m/z found, 278.1 [M+H]⁺.

### Example 67

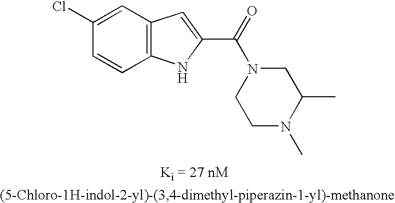

The product of Example 64, (5-Chloro-1H-indol-2-yl)-(3-methyl-piperazin-1-yl)-methanone (0.19 g) was dissolved in dichloromethane (10 mL). At room temperature, paraformaldehyde (0.031 g) was added, followed by acetic acid (1drop). The reaction mixture was stirred at ambient temperature for 5 h. Sodium triacetoxybrohydride (0.318 g) was added. The reaction mixture was stirred at ambient temperature for 16 h and poured into dichloromethane (20 mL), washed with water, saturated sodium hydrogencarbonate solution and then brine, dried over sodium sulfate, filtered, and concentrated under reduced pressure. The residue was purified via silica gel chromatography (0-10% methanol/dichloromethane) to give the title compound (0.22 g). ¹H NMR (400 MHz, CDCl₃): δ10.69 (br s, 1H), 7.56 (d, J=1.76 Hz, 1H), 7.33 (d, J=8.80 Hz, 1H), 7.16 (dd, J=1.96, 6.66 (d, J=1.57 Hz, 1H), 4.63-4.36 (m, 2H), 3.63-2.67 (m, 3H), 2.30 (s, 3H), 2.30-2.20 (m, 1H), 2.18-2.09 (m, 1H), 1.12 (d, J=5.87 Hz, 1H). MS (electrospray): exact mass calculated for C₁₅H₁₈ClN₃O, 291.11; m/z found, 292.1 [M+H]⁺.

The title compound of the following example (68) was prepared according to the general procedure of Scheme 5.

### Example 68

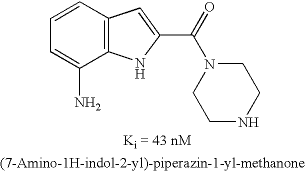

A mixture of 7-nitroindole-2-carboxylic acid (4.38 g) and 1-(3-dimethylaminopropyl)-3-ethylcarbodiimide hydrochloride (4.89 g) in dichloromethane (50 mL) was treated with piperazine-1-carboxylic acid tert-butyl ester (1.63 g) and stirred at ambient temperature for 16 h. The reaction mixture was poured into in dichloromethane (20 mL), washed with water, saturated sodium hydrogencarbonate solution and then brine, dried over sodium sulfate, filtered, and concentrated under reduced pressure. The residue was purified via silica gel chromatography (0-5% methanol/dichloromethane) to give 4-(7-Nitro-1H-indole-2-carbonyl)-piperazine-1-carboxylic acid tert-butyl ester (2.17 g). This material (1.69 g) was dissolved in CH₃OH (50 mL). At room temperature, ammonium formate (2.85 g) was added, followed by 10% palladium on carbon (0.47 g). The reaction mixture was heated to reflux for forty min, cooled and filtered through celite pad. The filtrate was concentrated and the residue was purified via silica gel chromatography (0-10% methanol/dichloromethane) to give 4-(7-Amino-1H-indole-2-carbonyl)-piperazine-1-carboxylic acid tert-butyl ester (1.34 g). This material (1.3 g) was treated with 20% trifluoroacetic acid/dichloromethane (50 mL) and stirred at ambient temperature for 1 h. The solvent was removed under reduced pressure to afford (7-Amino-1H-indol-2-yl)-piperazin-1-yl-methanone trifluoroacetate salt. This intermediate was dissolved in dichloromethane (100 mL), washed with saturated sodium hydrogencarbonate solution and then brine, dried over sodium sulfate, filtered, and concentrated under reduced pressure. The residue was purified via silica gel chromatography (0-10% 2M ammonia in methanol/dichloromethane) to give the title compound (0.824 g). ¹H NMR (400 MHz, CDCl₃/CD₃OD): δ7.09 (d, J=7.83 Hz, 1H), 6.95 (t, J=7.63 Hz, 1H), 6.72 (s, 1H), 6.60 (d, J=7.63 Hz, 1H), 4.20 (br s, 4H), 3.88 (br s, 4H), 2.94 (t, J=5.09 Hz, 3H). MS (electrospray): exact mass calculated for C₁₃H₁₆N₄O, 244.13; m/z found, 245.1 [M+H]⁺.

The title compounds of the following examples (69-70) were prepared according to the general procedure of Scheme 4.

### Example 69

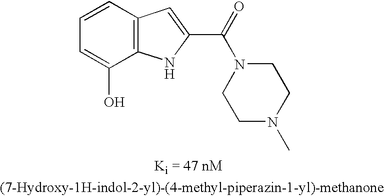

At room temperature, diethyl oxalate (13.6 mL) was added to a solution of potassium ethoxide (8.4 g) in anhydrous ethyl ether (200 mL). After 10 min, 3-methyl-2-nitroanisole (16.7 g) was added and stirred at ambient temperature for 24 h. The lumpy, deep purple potassium salt was separated by filtration and washed with anhydrous ether until the filtrate remained colorless. This salt was dissolved in aqueous ammonium chloride, and the solution was extracted with dichloromethane. The combined organic extracts were washed with brine, dried over sodium sulfate and filtered, and the solvent was evaporated. The residue was purified via silica gel chromatography (5-30% ethyl acetate/hexanes) to give 3-(3-Methoxy-2-nitro-phenyl)-2-oxo-propionic acid ethyl ester (14.0 g). This material (14.0 g) was dissolved in ethanol (200 mL) containing 5 wt. % palladium on activated carbon (1.4 g) and placed on a Parr hydrogenator at 60 psi H₂. After 2 h, the mixture was filtered through Celite, and concentrated to give a clear liquid. The liquid was purified by silica gel chromatography (5%-30% EtOAc/Hexanes) to obtain (7-Methoxy-1H-indol-2-yl)-(4-methyl-6-Methoxy-1H-indole-2-carboxylic acid ethyl ester (11.7 g). This ethyl ester (4.0 g) was treated with lithium hydroxide (1.75 g) in THF (100 mL) followed by water (30 mL) and stirred at ambient temperature for 16 h. The solution was acidified with 10% hydrochloric acid, diluted with water and extracted with ethyl acetate. The organic extracts were washed with brine, dried over sodium sulfate, filtered, and concentrated to afford 6-Methoxy-1H-indole-2-carboxylic acid (3.50 g). This material (3.50 g) and 1-(3-dimethylaminopropyl)-3-ethylcarbodiimide hydrochloride (5.26 g) in dichloromethane (100 mL) were treated with N-methylpiperazine (3.05 mL) and stirred at ambient temperature for 16 h. The reaction mixture was poured into dichloromethane (200 mL), washed with water, saturated sodium hydrogencarbonate solution and then brine, dried over sodium sulfate, filtered, and concentrated under reduced pressure. The residue was purified via silica gel chromatography (0-10% methanol/dichloromethane) to give (7-Methoxy-1H-indol-2-yl)-(4-methyl-piperazin-1-yl)-methanone (4.50 g). This material (3.5 g) was dissolved in dichloromethane (85 mL). At room temperature, 1 M Boron tribromide (2.42 mL) was added dropwise. The reaction mixture was heated to reflux for 2 h, cooled, and then quenched with saturated sodium hydrogencarbonate solution. The suspension was filtered. The filtrate was washed with saturated sodium hydrogencarbonate solution and then brine, dried over sodium sulfate and filtered, and solvent was evaporated. The residue was purified via silica gel chromatography (0-10% methanol/dichloromethane) to give the title compound (1.95 g). ¹H NMR (400 MHz, CDCl₃/CD₃OD): δ7.52 (s, 1H), 7.16 (dd, J=0.78, 7.24 Hz, 1H), 6.96 (t, J=7.63 Hz, 1H), 6.77 (s, 1H), 6.70 (dd, J=0.98, 6.65 Hz, 1H), 3.93 (br s, 4H), 2.55 (t, J=5.09 Hz, 4H), 2.38 (s, 3H). MS (electrospray): exact mass calculated for C₁₄H₁₇N₃O₂, 259.13; m/z found, 260.1 [M+H]⁺.

### Example 70

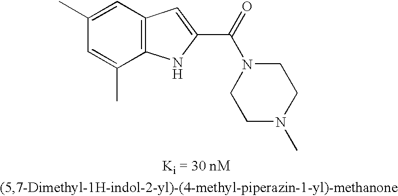

¹H NMR (400 MHz, CDCl₃): δ10.68 (br s, 1H), 7.20 (s, 1H), 6.80 (s, 1H), 6.65 (d, J=2.15 Hz, 1H), 3.91 (br s, 4H), 2.39 (t, J=4.50 Hz, 4H), 2.35 (s, 6H), 2.26 (s, 3H). MS (electrospray): exact mass calculated for C₁₆H₂₁N₃O, 271.17; m/z found, 272.1 [M+H]⁺.

### Example 71

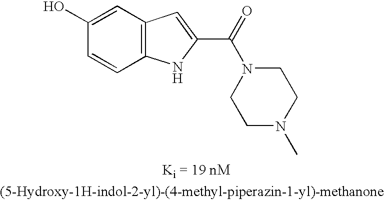

A mixture of the product of Example 5, (5-Benzyloxy-1H-indol-2-yl)-(4-methyl-piperazin-1-yl)-methanone (0.2 g) in a mixture of ethanol (3 mL) and ethylacetate (5 mL) was treated with 10% palladium on carbon (approximately 0.025 g ) and hydrogenated at atmospheric pressure for 2 h. The reaction mixture was filtered through a pad of Celite and the residue washed with methanol. The solvent in the combined filtrates was removed under reduced pressure, and the residue was purified via silica gel chromatography (3-10% 2M ammonia in methanol/dichloromethane) to afford the title compound (0.034 g, 23%). ¹H NMR (400 MHz, CD₃OD): δ7.20 (d, J=8.0 Hz, 1H), 6.90 (m, 1H), 6.75 (dd, J=4, 8 Hz, 1H), 6.54 (m, 1H), 3.80 (br.m, 4H), 2.44 (m, 4H), 2.27 (s, 3H). MS (electrospray): exact mass calculated for C₁₄H₁₇N₃O₂, 259.13; m/z found, 260.0 [M+H]⁺.

### Example 72

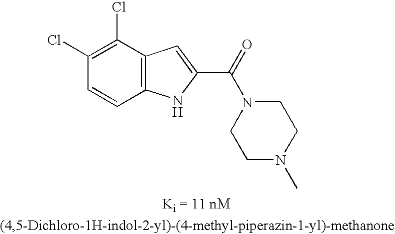

The title compound was prepared according to the general procedure of Scheme 2. A mixture of 3,4-dichlorophenylhydrazine (5.0 g) in benzene (50 mL) was treated sequentially with ethylpyruvate (2.6 mL) and p-toluenesulfonic acid (trace). The mixture was heated at reflux temperature (Dean and Stark conditions) for 5 h then cooled to ambient temperature to afford a solution of 2-[(3,4-dichloro-phenyl)-hydrazono]-propionic acid ethyl ester. Separately a solution of p-toluenesulfonic acid (15 g) in benzene (150 mL) was heated at reflux temperature (Dean and Stark conditions) for 2 h and then treated with the hydrazone solution. After 3 h the reaction mixture was cooled, treated with saturated sodium hydrogen carbonate solution and diethyl ether. The organic fraction was separated, washed with saturated sodium hydrogen carbonate solution and then brine, dried over magnesium sulfate and filtered, and solvent was evaporated to give an orange solid. The solid was purified via silica gel chromatography (15-75% ethylacetate/hexane) to afford 4,5-Dichloro-1H-indole-2-carboxylic acid ethyl ester (0.5 g, 8%) and 5,6-Dichloro-1H-indole-2-carboxylic acid ethyl ester (0.297 g, 5%). These materials were used separately without further purification. 4,5-Dichloro-1H-indole-2-carboxylic acid ethyl ester (0.5 g) was teated with 1M lithium hydroxide in ethanol (3 mL) and heated, water bath, for 2 h. The solution was acidified with 10% hydrochloric acid, diluted with water and extracted with ethylacetate. The organic extracts were combined, dried over sodium sulfate and filtered, and solvent was evaporated to give 4,5-dichloro-1H-indole-2-carboxylic acid (0.27 g, 60%). This material was treated with ethyl-3-(3′-dimethylaminopropyl)-carbodiimide hydrochloride (0.5 g), HOBT (0.4 g) and N, N-diisopropylmethyl mine (1 mL) in DMF (2 mL) and dichloromethane (2 mL) was treated with N-methylpiperazine (0.2 mL) stirred at ambient temperature for 18 h then diluted with water. The organic portion was separated, washed with brine, dried over sodium sulfate, and filtered. Solvent was removed under reduced pressure, and the residue was purified via silica gel chromatography (3-8% 2M ammonia in methanol/dichloromethane) to give the title compound (0.15 g, 40%). ¹H NMR (400 MHz, CDCl₃): δ10.2 (br.s, 1H), 7.25-7.16 (m, 2H), 6.75 (d, J=2 Hz, 1H), 3.92 (br.m, 4H), 2.47 (m, 4H), 2.30 (s, 3H). MS (electrospray): exact mass calculated for C₁₄H₁₅Cl₂N₃O, 311.06; m/z found, 312.0 [M+H]⁺.

### Example 73

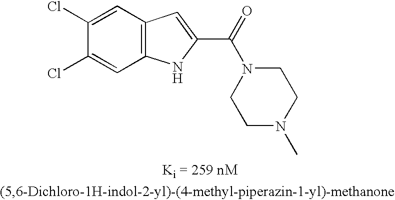

Using the procedure of the previous example (72), the title compound was prepared from 5,6-Dichloro-1H-indole-2-carboxylic acid ethyl ester. ¹H NMR (400 MHz, CDCl₃): δ9.9 (br.s, 1H), 7.97 (s, 1H), 7.79 (m, 1H), 6.94 (m, 1H), 4.20 (br.m, 4H), 2.77 (m, 4H), 2.26 (s, 3H). MS (electrospray): exact mass calculated for C₁₄H₁₅Cl₂N₃O, 311.06; m/z found, 312.0 [M+H]⁺.

The title compound of the following example (74) was prepared according to the general procedure of Scheme 5.

### Example 74

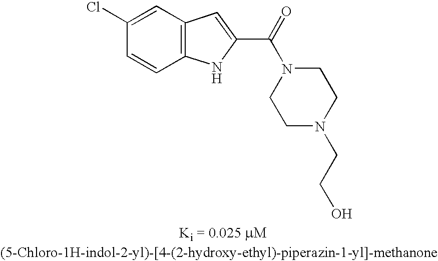

A. 4-(5-Chloro-1H-indole-2-carbonyl)-piperazine-1-carboxylic acid tert-butyl ester.

A mixture of 5-chloroindole-2-carboxylic acid (10 g), tert-butyl 1-piperazinecarboxylate (10.5 g) and 4-dimethylaminopyridine (6.3 g) in CH₂Cl₂ (200 mL) was treated with a catalytic amount of HOBT (0.2 g). The resulting mixture was cooled to 0° C., and EDCI (10.8 g) was added. The reaction was then slowly warmed to ambient temperature and stirred for 24 h then concentrated under reduced pressure. Water was added to the resulting residue. The product precipitated and was washed with water (2×50 mL) and Et₂O (30 mL). The resulting solid was dried under reduced pressure to yield (18.2 g). MS (electrospray): exact mass calculated for C₁₈H₂₂ClN₃Ohd 3, 363.13; m/z found, 362.3 [M-H]⁻.

B. (5-Chloro-1H-indol-2-yl)-piperazin-1-yl-methanone.

The product from Step A (11 g) was suspended in CH₂Cl₂ (75 mL), and TFA was added dropwise (75 mL). The resulting solution was stirred overnight at ambient temperature. The reaction solution was concentrated under reduced pressure, and the resulting residue was dissolved in CH₂Cl₂ (100 mL). Saturated aqueous NaHCO₃ (100 mL) was added slowly with stirring. After 20 min the organic layer was separated, washed with water (10 mL) and then brine (30 mL), and dried over Na₂SO₄. The organic layer was then concentrated under reduced pressure and purified via silica gel chromatography (0-35% methanol/dichloromethane) to give the title compound (7.6 g). MS (electrospray): exact mass calculated for C₁₃H₁₄ClN₃O, 263.08; m/z found, 264.1 [M+H]⁺.

C. (5-Chloro-1H-indol-2-yl)-[4-(2-hydroxy-ethyl) -piperazin-1-yl]-methanone.

The product from Step B (1.0 g) was dissolved in CH₃CN (10 mL) and treated with 2-bromoethanol (0.5 g) and then K₂CO₃ (0.8 g). The resulting mixture was heated at 60° C. overnight. The mixture was cooled to ambient temperature, filtered, and concentrated under reduced pressure. The resulting residue was purified via silica gel chromatography (0-10% methanol/dichloromethane) to give the title compound (0.5 g). ¹H NMR (400 MHz, CDCl₃): δ10.09 (br s, 1H), 7.61 (d, J=2.0 Hz, 1H), 7.37 (d, J=8.8 Hz, 1H), 7.23 (dd, J=2.0, 8.8 Hz, 1H), 6.69 (d, J=0.8 Hz, 1H), 3.95 (br m, 3H), 3.72-3.69 (m, 2H), 2.67-2.64 (m, 4H), 2.52 (br s, 3H). MS (electrospray): exact mass calculated for C₁₅H₁₈ClN₃O₂, 307.78; m/z found, 308.1 [M+H]⁺.

### Example 75

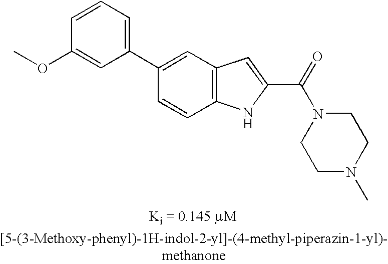

A suspension of (5-Bromo-1H-indol-2-yl)-(4-methyl-piperazin-1-yl)-methanone (Example 3, 0.057 g) in dry toluene (0.5 mL) was treated with Pd(OH)₂ (0.001 g) under N₂ atmosphere. The resulting mixture was then treated with 3-methoxyphenylboronic acid (0.057 g) and then K₃PO₄ (0.12 g), and heated at 95° C. for 24 h. The reaction mixture was cooled to ambient temperature and diluted with water (2 mL) and toluene (10 mL). The organic layer was separated and washed with brine (2 mL), dried over Na₂SO₄, filtered, and concentrated under reduced pressure. The resulting residue was purified via silica gel chromatography (0-12% methanol/dichloromethane) to give the title compound (0.005 g). ¹H NMR (400 MHz, CDCl₃): δ9.96 (br s, 1H), 7.53 (d, J=1.6 Hz, 1H), 7.51 (d, J=1.6 Hz, 1H), 7.35 (t, J=7.9 Hz, 1H), 7.23 (s, 1H), 7.21 (s, 1H), 7.17 (m, 1H), 6.87 (dd, J=2.2, 8.1 Hz, 1H), 6.82 (d, J=1.8 Hz, 1H), 3.99 (br s, 4H), 3.86 (s, 3H), 2.52 (t, J=4.9 Hz, 4H), 2.35 (s, 3H). MS (electrospray): exact mass calculated for C₂₁H₂₃N₃O₂, 349.18; m/z found, 350.2 [M+H]⁺.

The title compound of the following example (76) was prepared according to the general procedure of Example 75.

### Example 76

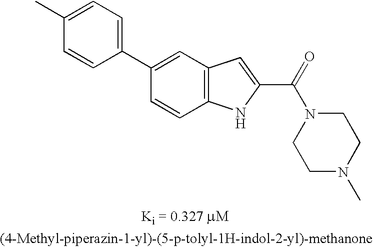

¹H NMR (400 MHz, CDCl₃): δ9.24 (br s, 1H), 7.81 (m, 1H), 7.54-7.46 (m, 5H), 7.26 (d, J=7.8 Hz, 1H), 6.82 (dd, J=0.7, 2.1 Hz, 1H), 3.97 (brs, 4H), 2.52 (t, J=5.1, 4H) 2.40 (s, 3H), 2.36 (s, 3H). MS (electrospray): exact mass calculated for C₂₁H₂₃N₃O, 333.18; m/z found, 334.2 [M+H]⁺.

### Example 77

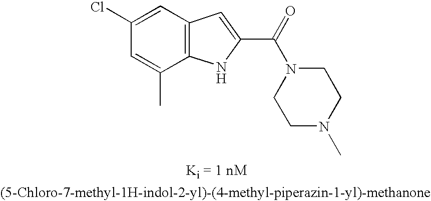

¹H NMR (400 MHz, CDCl₃): δ10.93 (br s, 1H), 7.39 (s, 1H), 6.93 (s, 1H), 6.67 (d, J=2.15 Hz, 1H), 3.92 (br s, 4H), 2.45 (t, J=4.89 Hz, 4H), 2.34 (s, 3H), 2.31 (s, 3H). MS (electrospray): exact mass calculated for C₁₅H₁₈ClN₃O, 291.11; m/z found, 292.1 [M+H]⁺.

### F. BIOLOGICAL EXAMPLES

### Example 1

Binding Assay on Recombinant Human Histamine H₄ Receptor

SK-N-MC cells or COS7 cells were transiently transfected with pH4R and grown in 150 cm² tissue culture dishes. Cells were washed with saline solution, scraped with a cell scraper and collected by centrifugation (1000 rpm, 5 min). Cell membranes were prepared by homogenization of the cell pellet in 20 mM Tris-HCl with a polytron tissue homogenizer for 10 s at high speed. Homogenate was centrifuged at 1000 rpm for 5 min at 4° C. The supernatant was then collected and centrifuged at 20,000×g for 25 min at 4° C. The final pellet was resuspended in 50 mM Tris-HCl. Cell membranes were incubated with ³H-histamine (5 nM -70 nM) in the presence or absence of excess histamine (10000 nM). Incubation occurred at room temperature for 45 min. Membranes were harvested by rapid filtration over Whatman GF/C filters and washed 4 times with ice cold 50 mM Tris HCI. Filters were then dried, mixed with scintillant and counted for radioactivity. SK-N-MC or COS7 cells expressing human histamine H₄ receptor were used to measure the affinity of binding of other compounds and their ability to displace ³H-ligand binding by incubating the above-described reaction in the presence of various concentrations of inhibitor or compound to be tested. For competition binding studies using ³H-histamine, Kᵢ values were calculated based on an experimentally determined K_{D} value of 5 nM and a ligand concentration of 5 nM according to Y.-C. Cheng and W. H. Prusoff where; Kᵢ=(IC₅₀)/(1+([L]/(K_{D})).

### Example 2

The Inhibition of Zymosan Induced Peritonitis in Mice by Histamine H4 Receptor Antagonists

This example demonstrates the discovery that histamine H₄ receptor antagonists can block the peritonitis induced by zymosan, which is the insoluble polysaccharide component on the cell wall of Saccharomyces cerevisiae. This is commonly used to induce peritonitis in mice and appears to act in a mast cell dependent manner.

Materials and Methods.

Animals

Male out-bred Swiss albino mice were purchased from Bantin and Kingman (T.O. strain; Hull, Humberside) and maintained on a standard chow pellet diet with tap water ad libitum and a 12:00 h light /dark cycle. All animals were housed for at least 3 days prior to experimentation to allow body weight to reach ˜30 g on the day of the experiment. For this particular experiment body weight was 30.5±0.3 g (n=32). Animals were briefly (30-60 s) anesthetized with halothane for all s.c. and i.p. treatments described below.

Drug Treatment and Experimental Design

Drugs were stored at room temperature, in the dark. On the day of the experiment, drugs were dissolved in sterile PBS as depicted below, and generously vortexed.

The compound from Chemical Example 1 was prepared at 10 mg/5 mL, and injected at 5 mL/kg. Imetit was prepared at 5 mg/5 mL, and injected at 5 mL/kg.

Thioperamide was prepared at 5 mg/5 mL, and injected at 5 mL/kg.

Time −15 min: Compounds or PBS administered s.c. at the reported doses.

Time 0: At time 0, mice received 1 mg zymosan A (Sigma) i.p.

Time +2 h: Compounds or PBS administered s.c. at the reported doses.

Time +4: Peritoneal cavities were washed 4 h later with 3 mL of PBS containing 3 mM EDTA, and the number of migrated leukocytes determined, by taking an aliquot (100 μL) of the lavage fluid and diluting 1:10 in Turk's solution (0.01% crystal violet in 3% acetic acid). The samples were then vortexed and 10 μL of the stained cell solution were placed in a Neubauer haemocytometer. Differential cell counts were performed using a light microscope (Olympus B061). In view of their chromatic characteristics and their nucleus and cytoplasm appearance, polymorphonuclear leukocytes (PMN; >95% neutrophils) could be easily identified.

Experimental groups are described below:

PBS +zymosan, n=8

Compound from Example 1+zymosan, n=8

Imetit+zymosan, n=8

Thioperamide+zymosan, n=8

Statistics

Data are shown for single mice, and also shown as mean ±SD or Standard Error (SE) of 8 mice per group. The % of inhibition is also shown. Statistical differences were determined by Anova followed by Bonferroni's post-hoc test.

Results

TABLE 1
Effect of compounds on zymosan peritonitis

| | | PMN | | | | P value |
|---|---|---|---|---|---|---|
| Treatment | n | (10⁶ per mouse) | mean | SD | SE | (% inhib) |
| PBS | 1 | 15.9 | 17.2 | 2.4 | 0.8 | |
| (s.c.) | 2 | 18.3 | | | | |
| | 3 | 16.2 | | | | |
| | 4 | 17.4 | | | | |
| | 5 | 19.8 | | | | |
| | 6 | 12.6 | | | | |
| | 7 | 19.8 | | | | |
| | 8 | 17.7 | | | | |
| Compound 1 | 1 | 9.9 | 6.6 | 2.7 | 1.0 | 0.001 |
| (10 mg/kg; s.c.) | 2 | 3.6 | | | | (−62%) |
| | 3 | 9.3 | | | | |
| | 4 | 3.3 | | | | |
| | 5 | 8.1 | | | | |
| | 6 | 5.1 | | | | |
| | 7 | 6.9 | | | | |
| Imetit | 1 | 19.8 | 17.3 | 2.6 | 0.9 | n.s. |
| (5 mg/kg; s.c.) | 2 | 17.1 | | | | — |
| | 3 | 14.1 | | | | |
| | 4 | 15.3 | | | | |
| | 5 | 21.3 | | | | |
| | 6 | 17.7 | | | | |
| | 7 | 14.1 | | | | |
| | 8 | 18.6 | | | | |
| Thioperamide | 1 | 9.3 | 9.3 | 3.4 | 1.2 | 0.001 |
| (5 mg/kg; s.c.) | 2 | 16.5 | | | | (−46%) |
| | 3 | 7.2 | | | | |
| | 4 | 10.8 | | | | |
| | 5 | 5.4 | | | | |
| | 6 | 9.9 | | | | |
| | 7 | 6.9 | | | | |
| | 8 | 8.1 | | | | |

From data analysis it can be seen that zymosan produced a leukocyte extravasation response that was intense at the 4 h time-point. Treatment with 10 mg/kg Compound 1 significantly reduced PMN influx (compare PBS group to Compound 1 group in Table 1). The degree of inhibition was >60%. Imetit (5 mg/kg) was inactive, whereas a significant inhibitory effect was attained by 5 mg/kg thioperamide.

Conclusion

To conclude, this study demonstrates that a histamine H₄ receptor antagonist, Compound 1, given at the dose of 10 mg/kg, is effective in reducing PMN accumulation in an experimental model of cell recruitment in response to local application of zymosan in the mouse peritoneal cavity. Furthermore thioperamide which is a dual H3/H₄ receptor antagonist is also effective. The dual H3/H₄ receptor agonist, Imetit, does not have any effect. This shows that an antagonist of the histamine H₄ receptor can block inflammation induced by zymosan.

### Example 3

The Inhibition of Sodium Urate Crystal Induced Peritonitis in Mice by histamine H₄ Receptor Antagonists

This example demonstrates the discovery for the first time that histamine H₄ receptor antagonists can block the peritonitis induced by sodium urate crystals. Such crystals are the primary cause of the inflammation associated with acute gouty arthritis.

Materials and Methods.

Animals

Male out-bred Swiss albino mice were purchased from Bantin and Kingman (T.O. strain; Hull, Humberside) and maintained on a standard chow pellet diet with tap water ad libitum and a 12:00 h light/dark cycle. All animals were housed for at least 3 days prior to experimentation to allow body weight to reach ˜30 g on the day of the experiment. For this particular experiment body weight was 30±1 (n=32).

Drug treatment and Experimental Design

Compound 1 was stored at room temperature in the dark. On the day of the experiment, Compound 1 was dissolved in phosphate buffered saline (PBS) to a concentration of 3 mg/mL. At time −15 min Compound 1 was administered s.c. at the dose of 10 mg/kg, whereas the control group received the vehicle alone (10 mL/kg). Mice received 3 mg mono sodium urate crystals (MSU) given intra-peritoneally at time 0. At time +2 h and time +4 h, Compound 1 (10 mg/kg) or vehicle (10 mL/kg) were given s.c.

Time +6 h: Peritoneal cavities were washed 6 h later with 3 mL of PBS containing 3 mM EDTA, and the number of migrated leukocytes determined, by taking an aliquot (100 μL) of the lavage fluid and diluting 1:10 in Turk's solution (0.01% crystal violet in 3% acetic acid). The samples were then vortexed and 10 μL of the stained cell solution were placed in a Neubauer hematocytometer. Differential cell counts were performed using a light microscope (Olympus B061). In view of their chromatic characteristics and their nucleus and cytoplasm appearance, cells polymorphonuclear cells (PMN, >95% neutrophils) could be easily differentiated

Experimental groups are described below:

Vehicle+MSU crystals n=8

Compound 1+MSU crystals n=8

Statistics

Data are shown for single mice, and also shown as mean ±SE of (n) mice per group. Statistical differences were determined by Student's t test. A P value <0.05 was taken as significant.

Results

TABLE 2
Effect of Compound 1 on MSU-induced leukocyte migration
as evaluated at the 6 h time-point.

| | | PMN | | | | P value |
|---|---|---|---|---|---|---|
| Treatment | n | (10⁶ per mouse) | mean | SD | SE | (% inhib) |
| PBS | 1 | 9.6 | 8.9 | 2.2 | 0.8 | |
| (s.c.) | 2 | 12.9 | | | | |
| | 3 | 7.2 | | | | |
| | 4 | 9.9 | | | | |
| | 5 | 6.6 | | | | |
| | 6 | 7.2 | | | | |
| | 7 | 10.5 | | | | |
| | 8 | 7.5 | | | | |
| Compound 1 | 1 | 7.8 | 6.8 | 2.1 | 0.7 | 0.04 |
| (10 mg/kg; s.c.) | 2 | 4.5 | | | | (−24%) |
| | 3 | 3.0 | | | | |
| | 4 | 7.8 | | | | |
| | 5 | 8.1 | | | | |
| | 6 | 9.3 | | | | |
| | 7 | 6.6 | | | | |
| | 8 | 7.2 | | | | |

Mice were treated with either PBS (10 mL/kg) or Compound 1 (10 mg/kg) at −15 min, +2 h and +4 h, and with 3 mg MSU crystals at time 0. PMN influx into the peritoneal cavity was measured at the 6 h time-point after collection of the lavage fluids and specific staining as described in the experimental section.

Conclusion

As expected, MSU crystals produced a PMN extravasation that was intense at the 6 h time-point. Treatment with a specific histamine H₄ receptor antagonist, Compound 1, significantly reduced PMN migration (Table 2): the degree of inhibition was 24%. To conclude, this study demonstrates that a histamine H₄ receptor antagonist is effective in reducing PMN accumulation in an experimental model of cell recruitment in response to local application of MSU crystals in the mouse peritoneal cavity.

### Example 4

The Inhibition of Croton Oil Induced Topical Inflammation in Mice by Histamine H₄ Receptor Antagonists

This example demonstrates the discovery that histamine H₄ receptor antagonists can block the inflammation associated with topical application of croton oil.

Materials and Methods.

Animals

Male or female ICR derived mice weighing 22±1 g were used. Space allocation for 5 animals was 45×23×15 cm. Mice were housed in APEC R cages. All animals were maintained in a controlled temperature (22° C. -24° C.) and humidity (60% -80%) environment with 12 h light/dark cycles. Free access to standard lab chow for Mice (LabDiet Rodent Diet, PMI Nutrition International, USA) and tap water was granted.

Chemicals

Acetone (Wako, Japan), Croton oil (Sigma,USA), Indomethacin (Sigma, USA) and Pyrogen free saline (Astar, Taiwan).

Protocol Croton Oil Induced Topical Inflammation

Groups of 5 ICR derived male mice weighing 22±1 g were used. Compound 1 (10 mg/kg) and vehicle (0.9% NaCl) as well as the positive control Indomethacin (30 mg/kg) were administered subcutaneously to test animals at 30 min before, and 2 and 4 h after croton oil (8% in 20 μL acetone) was applied topically. Ear swelling was measured by Dyer model micrometer gauge 6 h after croton oil as an index of inflammation.

Results

TABLE 3
Effect of Compound 1 on Croton Oil Induced Topical
Inflammation

| | | Difference in ear | | | P value |
|---|---|---|---|---|---|
| Treatment | n | Thickness (× 0.01 mm | Mean | SE | (% inhib) |
| PBS | 1 | 12 | 16.6 | 1.4 | |
| (s.c.) | 2 | 17 | | | |
| | 3 | 15 | | | |
| | 4 | 19 | | | |
| | 5 | 20 | | | |
| Compound 1 | 1 | 12 | 12.0 | 1.2 | 0.03 |
| (10 mg/kg; s.c.) | 2 | 10 | | | (−28%) |
| | 3 | 13 | | | |
| | 4 | 9 | | | |
| | 5 | 16 | | | |
| Indomethacin | 1 | 5 | 10.0 | 1.3 | 0.001 |
| (30 mg/kg; s.c.) | 2 | 10 | | | (−40%) |
| | 3 | 12 | | | |
| | 4 | 12 | | | |
| | 5 | 11 | | | |

Conclusion

In the croton oil induced topical inflammation ear swelling assay, a histamine H₄ receptor antagonist, Compound 1, at a dose of 10 mg/kg×3 (s.c.) significantly reduced the swelling with respect to the vehicle control. This effect was similar to Indomethacin (30 mg/kg×3). These results show that a histamine H₄ receptor antagonist can act as an anti-inflammatory agent.

### Example 5

Cell-type Distribution of H₄ Expression

RNA was prepared from the different cells using an RNeasy kit (Qiagen, Valencia, CA) according to the manufacturer's instructions. RNA samples (5 μg) were run on an RNA gel and then transferred overnight to a nylon blot (Hybond, Amersham Pharmacia Biotech, Piscataway, N.J.). The blot was pre-hybridized with ExpressHyb solution (CLONTECH) for 30 min at 68° C. The H₄ receptor DNA was labeled using the rediprime 11 kit (Amersham Pharmacia Biotech). The blot was hybridized for 2 h at 68° C., followed by one wash step (23 SSC and 0.05% SDS) of 40 min at room temperature, and a second wash step (0.13 SSC and 0.1% SDS) of 40 min at 50° C. The blot was exposed to X-ray film at 27° C. with two intensifying screens overnight.

Conclusion

The Northern Blot results indicate that the H₄ receptor is expressed on bone-marrow derived mast cells (BMMC) peritoneal mast cells, and eosinophils. These positive results are consistent with the published literature (eg. Oda et al., Nguyen et al., and Morse et al. in the Background section). However, the negative results of the Northern Blot experiment, such as the finding of apparently no measurable levels of H₄ receptor expressed by neutrophils, differ somewhat from the above literature findings. This may be explained by the different methodologies used. Additional investigation may also clarify these issues.

TABLE 4
Cell-type Distribution of H₄ Expression by Northern Blot

| Species | Cell Type | H₄ |
|---|---|---|
| Human | Eosinophils | + |
| | Immature Dendritic Cells | − |
| | Mature Dendritic Cells | − |
| | CD14⁺ Monocytes | − |
| | CD4⁺ T Cells | − |
| | CD8⁺ T Cells | − |
| | B Cells | − |
| | Neutrophils | − |
| Mouse | Eosinophils | + |
| | Peritoneal Mast Cells | + |
| | BMMC | + |
| | BM Derived Macrophages | − |
| | Peritoneal Macrophages | − |
| | CD4⁺ T Cells | − |
| | B Cells | − |

G. Other Embodiments

The features and advantages of the invention are apparent to one of ordinary skill in the art. Based on this disclosure, including the summary, detailed description, background, examples, and claims, one of ordinary skill in the art will be able to make modifications and adaptations to various conditions and usages. Publications described herein are incorporated by reference in their entirety. These other embodiments are also within the scope of the invention.

What is claimed is:

1. A method for treating allergic rhinitis in a patient, said method comprising administering to the patient a pharmaceutically effective amount of a composition comprising a compound of formula (I): 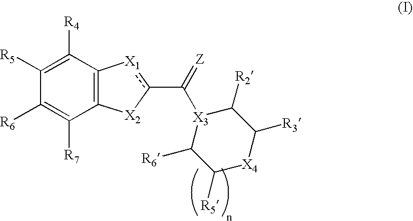
Wherein R₁ is Rₐ, RₐR_{b}—, Rₐ—O—R_{b}—, or (R_{c})(R_{d})N—R_{b}—, where Rₐ is H, cyano, —(C═O)N(R_{c})(R_{d}), —C(═NH)(NH₂), C₁₋₁₀ alkyl, C₃₋₈ alkenyl, C₃₋₈ cycloalkyl, C₂₋₅ heterocyclic radical, or phenyl; where R_{b} is C₁₋₈ alkylene, C₂₋₈ alkenylene, C₃₋₈ cycloalkylene, bivalent C₃₋₈ heterocyclic radical, or phenylene; and R_{c} and R_{d} are each independently H, C₁₋₈ alkyl, C₂₋₈ alkenyl, C₃₋₈ cycloalkyl, or phenyl;
R_{2′} is H, methyl, ethyl, NRₚR_{q}, —(CO)NRₚR_{q}, —(CO)ORᵣ, —CH₂NRₚR_{q}, or CH₂ORᵣ; where Rₚ, R_{q}, and Rᵣ are independently selected from C₁₋₆ alkyl, C₃₋₆ cycloalkyl, phenyl; (C₃₋₆ cycloalkyl)(C₁₋₂ alkylene), benzyl or phenethyl; or Rₚ and R_{q} taken together with the nitrogen to which they are attached, form a 4-7 membered heterocyclic ring with 0 or 1 additional heteroatoms selected from O, S, and N;
R_{3′} is H, methyl, ethyl, NRₛRₜ, —(CO)NRₛRₜ, —(CO)ORᵤ, —CH₂NRₛRₜ, or CH₂ORᵤ; where Rₛ, Rₜ, and Rᵤ are independently selected from C₁₋₆ alkyl, C₃₋₆ cycloalkyl, phenyl; (C₃₋₆ cycloalkyl)(C₁₋₂ alkylene), benzyl or phenethyl; or Rₛ and Rₜ taken together with the nitrogen to which they are attached, form a 4-7 membered heterocyclic ring with 0 or 1 additional heteroatoms selected from O, S, and N;
R_{5′} is methyl, ethyl, or H;
R_{6′} is methyl, ethyl, or H;
R_{7′} is methyl, ethyl, or H;
X₄ is NR₁ or S;
X_{1 is CR}₃;
R₃ is F, Cl, Br, CHO, R_{f}, R_{f}R_{g}—, R_{f}—O—R_{g}—, or (Rₕ)(Rᵢ)N—R_{g}—, where R_{f} is H, C₁₋₆ alkyl, C₂₋₆ alkenyl, C₃₋₆ cycloalkyl, C₂₋₅ heterocyclic radical, or phenyl; where R_{g} is C₁₋₆ alkylene, C₂₋₆ alkenylene, C₃₋₆ cycloalkylene, bivalent C₃₋₆ heterocyclic radical, or phenylene; and Rₕ and Rᵢ are each independently H, C₁₋₆ alkyl, C₂₋₆ alkenyl, C₃₋₆ cycloalkyl, or phenyl;
X₂ is NRₑ or O; Rₑ is H or C₁₋₆ alkyl;
X₃ is N;
Z is ═O or ═S;
each of R₄ and R₆ is independently H, F, Cl, Br, I, COOH, OH, nitro, amino, cyano, C₁₋₄ alkoxy, or C₁₋₄ alkyl;
R₅ is H, F, Cl, Br, I, (C═O)Rⱼ, OH, nitro, NRⱼRₖ, cyano, phenyl, —OCH₂—Ph, C₁₋₄ alkoxy, or C₁₋₄ alkyl;
R₇ is H, F, Cl, Br, I, (C═O)Rₘ, OH, nitro, NRₗRₘ, cyano, phenyl, —OCH₂—Ph C₁₋₄ alkoxy, or C₁₋₄ alkyl;
wherein each of Rⱼ, Rₖ, Rₗ, and Rₘ is independently selected from H, C₁₋₆ alkyl, hydroxy, phenyl, benzyl, phenethyl, and C₁₋₆ alkoxy;
each of the above hydrocarbyl (including alkyl, alkoxy, phenyl, benzyl, cycloalkyl, and so on) or heterocyclic groups being independently and optionally substituted with between 1 and 3 substituents selected from C₁₋₃ alkyl, halo, hydroxy, amino, and C₁₋₃ alkoxy;
wherein n is 0, 1, or 2; where n is 2, the moiety —(CHR_{5′})ₙ₌₂— is —(CHR_{5′}—CHR_{7′})— where CHR_{5′} is between CHR_{6′} and CHR_{7′};
provided at least one of R₁, R_{2′}, R₃, R₄, R₅, R₆, and R₇ is other than H when Z is O;
and provided, where Z is O, n=1, and each of R₄, R₅, R₆, R₇, R_{2′}, R_{3′}, R_{5′}, and R_{6′} is H, then (a) where X₂ is NH, then R₁ is (i) not methyl, pyridyl, phenyl, or benzyl, and (b) where X₂ is O, then R₁ is not methyl;
and provided, where Z is O, X₂ is NH, n=1, R₁ is methyl, each of R₄, R₆, R₇, R_{2′}, R_{3′}, R_{5′}, and R_{6′} is H, then R₅ is not methoxy;
or a pharmaceutically acceptable salt, ester, or amide thereof.

2. The method of claim 1 wherein said composition comprises a compound of the formula: 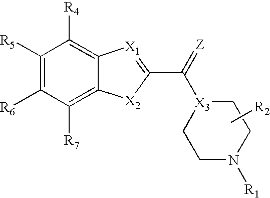
Wherein R₁ is Rₐ, RₐR_{b}—, Rₐ—O—R_{b}—, or (R_{c})(R_{d})N—R_{b}—, where Rₐ is H, C₁₋₁₀ alkyl, C₃₋₈ alkenyl, C₃₋₈ cycloalkyl, C₂₋₅ heterocyclic radical, or phenyl; where R_{b} is C₁₋₈ alkylene, C₃₋₈ alkenylene, C₃₋₈ cycloalkylene, bivalent C₃₋₈ heterocyclic radical, or phenylene; and R_{c} and R_{d} are each independently H, C₁₋₈ alkyl, C₂₋₈ alkenyl, C₃₋₈ cycloalkyl, or phenyl;
R₂ is ortho or meta, and is methyl or H;
X₁ is CR₃;
R₃ is F, Cl, Br, R_{f}, R_{f}R_{g}—, R_{f}—O—R_{g}—, or (Rₕ)(Rᵢ)N—R_{g}—, where Rf is H, C₁₋₆ alkyl, C₂₋₆ alkenyl, C₃₋₆ cycloalkyl, C₂₋₅ heterocyclic radical, or phenyl; where R_{g} is C₁₋₆ alkylene, C₂₋₆ alkenylene, C₃₋₆ cycloalkylene, bivalent C₃₋₆ heterocyclic radical, or phenylene; and Rₕ and Rᵢ are each independently H, C₁₋₆ alkyl, C₂₋₆ alkenyl, C₃₋₆ cycloalkyl, or phenyl;
X₂ is NRₑ or O; Rₑ is H or C₁₋₆ alkyl;
X₃ is N;
Z is ═O or ═S;
each of R₄ and R₆ is independently H, F, Cl, Br, I, COOH, OH, nitro, amino, cyano, C₁₋₄ alkoxy, or C₁₋₄ alkyl;
R₅ is H, F, Cl, Br, I, (C═O)Rⱼ, OH, nitro, NRⱼRₖ, cyano, —OCH₂—Ph, C₁₋₄ alkoxy, or C₁₋₄ alkyl;
R₇ is H, F, Cl, Br, I, (C═O)Rₘ, OH, nitro, NRₗRₘ, cyano, C₁₋₄ alkoxy, or C₁₋₄ alkyl;
wherein each of Rⱼ, Rₖ, Rₗ, and Rₘ is independently selected from H, C₁₋₆ alkyl, hydroxy, phenyl, benzyl, phenethyl, and C₁₋₆ alkoxy;
each of the above hydrocarbyl or heterocyclic groups being independently and optionally substituted with between 1 and 3 substituents selected from C₁₋₃ alkyl, halo, hydroxy, amino, and C₁₋₃ alkoxy;
provided at least one of R₁, R₂, R₃, R₄, R₅, R₆, and R₇ is other than H when Z is O;
or a pharmaceutically acceptable salt, ester, or amide thereof.

3. The method of claim 1 wherein said composition comprises a compound wherein R₁ is Rₐ, RₐR_{b}—, Rₐ—O—R_{b}—, or (R_{c})(R_{d})N—R_{b}—, where Rₐ is H, C₁₋₁₀ alkyl, C₂₋₅ alkenyl, C₃₋₈ cycloalkyl, C₂₋₅ heterocyclic radical, or phenyl; where R_{b} is C₁₋₆ alkylene, or C₂₋₈ alkenylene; and R_{c} and R_{d} are each independently H, C₁₋₈ alkyl, C₂₋₈ alkenyl, C₃₋₈ cycloalkyl, or phenyl;
R_{2′} is methyl or H;
R_{3′} is methyl or H;
R_{5′} is methyl or H;
R_{6′} is methyl or H;
R_{7′} is methyl or H;
X₁ is CR₃;
R₃ is F, Cl, Br, methyl, ethyl, or propyl;
X₂ is NRₑ or O; Rₑ is H or C₁₋₆alkyl;
X₃ is N;
Z is ═O or ═S;
each of R₄ and R₆ is independently H, F, Cl, Br, I, COOH, OH, nitro, amino, cyano, C₁₋₃ alkoxy, or C₁₋₃ alkyl;
R₅ is H, F, Cl, Br, I, (C═O)Rⱼ, OH, nitro, NRⱼRₖ, cyano, —OCH₂—Ph, C₁₋₄ alkoxy; or C₁₋₄ alkyl;
R₇ is H, F, Cl, Br, I, (C═O)Rₘ, OH, nitro, NRₗRₘ, cyano, C₁₋₄ alkoxy, or C₁₋₄ alkyl;
wherein each of Rⱼ, Rₖ, Rₗ, and Rₘ is independently selected from H, C₁₋₆ alkyl, hydroxy, phenyl, benzyl, phenethyl, and C₁₋₆ alkoxy;
each of the above hydrocarbyl or heterocyclic groups being independently and optionally substituted with between 1 and 3 substituents selected from C₁₋₃ alkyl, halo, hydroxy, amino, and C₁₋₃ alkoxy;
n is 1;
provided at least one of R₁, R_{2′}, R₃, R₄, R₅, R₆, and R₇ is other than H when Z is O;
or a pharmaceutically acceptable salt, ester, or amide thereof.

4. The method of claim 1 wherein said composition comprises a compound wherein
R₁ is H, methyl, or ethyl;
One of R_{2′} and R_{3′} is methyl, and the other is H, where R₁ is H; R₂ is otherwise H;
X₁ is CR₃; R₃ is H, F, Cl, or Br;
X₂ is NRₑ or O;
Rₑ is H or C₁₋₃ alkyl;
Z is ═O or ═S;
each of R₄ and R₆ is independently H, OH, C₁₋₄ alkyl, C₁₋₄ alkoxy, cyano, or amino;
R₅ is H, F, Cl, Br, COOH, OH, amino, cyano, C₁₋₄ alkoxy, or C₁₋₄ alkyl; and
R₇ is H, F, Cl, Br, C₁₋₄ alkyl, C₁₋₄ alkoxy, cyano, or amino; provided at least one of R₅ and R₇ is not H.

5. The method of claim 1 wherein said composition comprises a compound wherein
R₁ is H, methyl, or ethyl;
R_{2′} and R_{3′} are independently methyl or H;
X₁ is CR₃ or N; R₃is H, F, or Cl;
X₂ is NRₑ or O; Rₑ is H or C₁₋₆alkyl;
Z is ═O or ═S;
each of R₄ and R₆ is H;
R₅ is H, F, Cl, Br, methyl, ethyl, or propyl; and
R₇ is H, F, Cl, Br, or C₁₋₄ alkyl; provided at least one of R₅ and R₇ is not H.

6. The method of claim 1 wherein said composition comprises a compound wherein X₂ is N.

7. The method of claim 1 wherein said composition comprises a compound wherein X₂ is O.

8. The method of claim 1 wherein said composition comprises a compound wherein R₁ is H, methyl or ethyl.

9. The method of claim 1 wherein said composition comprises a compound wherein R₁ is methyl.

10. The method of claim 1 wherein said composition comprises a compound wherein R_{2′} is H.

11. The method of claim 1 wherein said composition comprises a compound wherein R_{2′} is methyl.

12. The method of claim 1 wherein said composition comprises a compound wherein R₃ is H or Cl.

13. The method of claim 12 wherein said composition comprises a compound wherein R₃ is Cl.

14. The method of claim 1 wherein said composition comprises a compound wherein R₅ is F, Cl, Br, or methyl and R₇ is F, Cl, or Br.

15. The method of claim 1 wherein said composition comprises a compound wherein each of R₅ and R₇ is independently selected from H, F, Cl, Br, and methyl, provided at least one of R₅ and R₇ is not H.

16. The method of claim 1 wherein said composition comprises a compound wherein each of R₄ and R₆ is independently H, methyl, or Cl.

17. The method of claim 1 wherein said composition comprises a compound wherein R₃ is H or Cl; R₅ is F, Cl, Br, or methyl; and R₇ is H, F, Cl, or Br.

18. The method of claim 17 wherein said composition comprises a compound wherein each of R₄ and R₆ is independently H, methyl, or Cl.

19. The method of claim 1 wherein said composition comprises a compound wherein Z is ═S.

20. The method of claim 1 wherein said composition comprises a compound selected from: (5-Chloro-1H-indol-2-yl)-(4-methyl-piperazin-1-yl)-methanone; (5-Fluoro-1H-indol-2-yl)-(4-methyl-piperazin-1-yl)-methanone; (5-Bromo-1H-indol-2-yl)-(4-methyl-piperazin-1-yl)-methanone; (5-Methyl-1H-indol-2-yl)-(4-methyl-piperazin-1-yl)-methanone; (5,7-Difluoro-1H-indol-2-yl)-(4-methyl-piperazin-1-yl)-methanone; (7-Chloro-1H-indol-2-yl)-(4-methyl-piperazin-1-yl)-methanone; (5,7-Dichloro-1H-indol-2-yl)-(4-methyl-piperazin-1-yl)-methanone; (5-Chloro-7-methyl-1H-indol-2-yl)-(4-methyl-piperazin-1-yl)-methanone; and (3,5-Dichloro-1H-indol-2-yl)-(4-methyl-piperazin-1-yl)-methanone.

21. The method of claim 1 wherein said composition comprises a compound selected from: (6-Chloro-1H-indol-2-yl)-(4-methyl-piperazin-1-yl)-methanone; (1H-Indol-2-yl)-(3-methyl-piperazin-1-yl)-methanone; (7-Bromo-1H-indol-2-yl)-(4-methyl-piperazin-1-yl)-methanone; (5-Bromo-benzofuran-2-yl)-(4-methyl-piperazin-1-yl)-methanone; and (1H-Indol-2-yl)-(4-methyl-piperazin-1-yl)-methanethione.

22. The method of claim 1 wherein said composition comprises a compound selected from: (5-Chloro-1H-indol-2-yl)-(4-methyl-piperazin-1-yl)-methanone; (5-Bromo-1H-indol-2-yl)-(4-methyl-piperazin-1-yl)-methanone; (5-Methyl-1H-indol-2-yl)-(4-methyl-piperazin-1-yl)-methanone; (5,7-Difluoro-1H-indol-2-yl)-(4-methyl-piperazin-1-yl)-methanone; and (5,7-Dichloro-1H-indol-2-yl)-(4-methyl-piperazin-1-yl)-methanone.

23. The method of claim 1 wherein said composition comprises a compound selected from:
(4-Methyl-piperazin-1-yl)-(5-trifluoromethyl-1H-indol-2-yl)-methanone; (7-Amino-5-methyl-1H-indol-2-yl)-(4-methyl-piperazin-1-yl)-methanone; (5-Amino-7-methyl-1H-indol-2-yl)-(4-methyl-piperazin-1-yl)-methanone; (7-Amino-5-bromo-1H-indol-2-yl)-(4-methyl-piperazin-1-yl)-methanone; (5-Amino-7-bromo-1H-indol-2-yl)-(4-methyl-piperazin-1-yl)-methanone; (5-Fluoro-7-methyl-1H-indol-2-yl)-(4-methyl-piperazin-1-yl)-methanone; (7-Fluoro-5-methyl-1H-indol-2-yl)-(4-methyl-piperazin-1-yl)-methanone; (6-Bromo-5-hydroxy-1H-indol-2-yl)-(4-methyl-piperazin-1-yl)-methanone; (5-Bromo-6-hydroxy-1H-indol-2-yl)-(4-methyl-piperazin-1-yl)-methanone; (6-Bromo-7-hydroxy-1H-indol-2-yl)-(4-methyl-piperazin-1-yl)-methanone; (4-Bromo-7-hydroxy-1H-indol-2-yl)-(4-methyl-piperazin-1-yl)-methanone; (6-Bromo-7-methyl-1H-indol-2-yl)-(4-methyl-piperazin-1-yl)-methanone; and (4-Bromo-7-methyl-1H-indol-2-yl)-(4-methyl-piperazin-1-yl)-methanone.

24. The method of claim 1 wherein said composition comprises a compound selected from: (5,7-Dichloro-1H-indol-2-yl)-piperazin-1-yl-methanone; (5,7-Difluoro-1H-indol-2-yl)-piperazin-1-yl-methanone; (5,7-Difluoro-1H-indol-2-yl)-(3-methyl-piperazin-1-yl)-methanone; (5,6-Difluoro-1H-indol-2-yl)-(4-methyl-piperazin-1-yl)-methanone; and (4,6-Difluoro-1H-indol-2-yl)-(4-methyl-piperazin-1-yl)-methanone.

25. The method of claim 1 wherein said composition comprises a compound selected from:
1-(5-Chloro-1H-indole-2-carbonyl)-4-methyl-piperazine-2-carboxylic acid methyl ester; 4-(5-Chloro-1H-indole-2-carbonyl)-1-methyl-piperazine-2-carboxylic acid methyl ester; 4-(5-Chloro-1H-indole-2-carbonyl)-1-methyl-piperazine-2-carboxylic acid amide; 1-(5-Chloro-1H-indole-2-carbonyl)-4-methyl-piperazine-2-carboxylic acid amide; 4-(5-Chloro-1H-indole-2-carbonyl)-1-methyl-piperazine-2-carboxylic acid methylamide; 1-(5-Chloro-1H-indole-2-carbonyl)-4-methyl-piperazine-2-carboxylic acid methylamide; 4-(5-Chloro-1H-indole-2-carbonyl)-1-methyl-piperazine-2-carboxylic acid dimethylamide; 1-(5-Chloro-1H-indole-2-carbonyl)-4-methyl-piperazine-2-carboxylic acid dimethylamide; (5-Chloro-1H-indol-2-yl)-(3-hydroxymethyl-4-methyl-piperazin-1-yl)-methanone; (5-Chloro-1H-indol-2-yl)-(3-methoxymethyl-4-methyl-piperazin-1-yl)-methanone; (5-Chloro-1H-indol-2-yl)-(2-methoxymethyl-4-methyl-piperazin-1-yl)-methanone; (5-Chloro-1H-indol-2-yl)-(4-methyl-3-methylaminomethyl-piperazin-1-yl)-methanone; (5-Chloro-1H-indol-2-yl)-(4-methyl-2-methylaminomethyl-piperazin-1-yl)-methanone; (5-Chloro-1H-indol-2-yl)-(3-dimethylaminomethyl-4-methyl-piperazin-1-yl)-methanone; and (5-Chloro-1H-indol-2-yl)-(2-dimethylaminomethyl-4-methyl-piperazin-1-yl)-methanone.

26. The compound (5-Chloro-7-methyl-1H-indol-2-yl)-(4-methyl-piperazin-1-yl)-methanone.
